(19) United States
(12) Patent Application Publication
(10) Pub. No.: US 2025/0064813 A1
(43) Pub. Date: Feb. 27, 2025

(54) STABLE LIQUID PHARMACEUTICAL FORMULATION

(71) Applicant: GENUV Inc., Seoul (KR)

(72) Inventors: Gabriel Arturo CORREIA ONTIVEROS, Utrecht (NL); Dae Il YEOM, Seoul (KR)

(73) Assignee: GENUV Inc., Seoul (KR)

(21) Appl. No.: 18/943,857

(22) Filed: Nov. 11, 2024

Related U.S. Application Data
(63) Continuation of application No. PCT/KR2023/006489, filed on May 12, 2023.

(30) Foreign Application Priority Data
May 12, 2022 (KR) 10-2022-0058197

(51) Int. Cl. A61K 31/519; A61K 9/00; A61K 9/107; A61K 9/48; A61K 47/10; A61K 47/14; A61K 47/22; A61K 47/24; A61K 47/26; A61K 47/32

(52) U.S. Cl. CPC A61K 31/519; A61K 9/0056; A61K 9/107; A61K 9/4858; A61K 9/4866; A61K 47/10; A61K 47/14; A61K 47/22; A61K 47/24; A61K 47/26; A61K 47/32

(57) ABSTRACT

The present invention relates to a stable liquid pharmaceutical formulation of a trametinib compound. According to the present invention, provided is a stable liquid pharmaceutical formulation suitable for oral administration, the stable liquid pharmaceutical formulation comprising: trametinib or a pharmaceutically acceptable salt or solvate thereof; and a pharmaceutically acceptable solubilizing agent. The liquid pharmaceutical formulation according to the present invention can be stored at room temperature, is stable even when stored for a long period of time, and can exhibit excellent dissolution characteristics upon administration, even when confronted with changes in the environment inside the gastrointestinal tract, and thus can improve drug absorption.

### CROSS-REFERENCE TO RELATED APPLICATION

This application is a continuation of the International PCT application serial no. PCT/KR2023/006489, filed on May 12, 2023, which claims priority of Korea application serial no. 10-2022-0058197, filed on May 12, 2022. The entirety of the above mentioned patent applications is hereby incorporated by reference herein and made a part of this specification.

### BACKGROUND

### 1. Technical Field

The present invention relates to stable liquid pharmaceutical formulation comprising trametinib. Specifically, the present invention relates to stable liquid pharmaceutical formulation particularly suitable for oral administration comprising trametinib or a pharmaceutically acceptable salt or solvate thereof and a pharmaceutically acceptable solubilizer(s).

### 2. Related Art

Trametinib is represented by the chemical formula below, and its chemical name is N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1 (2H)-yl}phenyl) acetamide (hereinafter referred to as “Compound 1”).

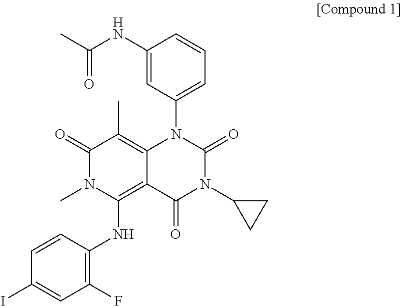 [SMILES: CC(=O)Nc1cccc(-n2c(=O)n(C3CC3)c(=O)c3c(Nc4ccc(I)cc4F)n(C)c(=O)c(C)c32)c1]

Compound 1 is a MEK 1/2 inhibitor, which inhibits both MEK1 and MEK2, the upstream components of ERK in the MAPK/ERK (mitogen-activated protein kinase/extracellular regulated kinase) signal transduction pathway. It is commercially available under the tradename Mekinist® or Meqsel® and is used as a cancer drug for melanoma, non-small cell cancer and the like. Recently, Compound 1 was presented as a therapeutic candidate effective for the treatment of neurodegenerative diseases such as Alzheimer's disease and amyotrophic lateral sclerosis (ALS) (WO 2018/097628, WO 2020/235954, WO 2021/210897). Specifically, Compound 1 induces neurogenesis by differentiating neural stem cells to neurons and protects neurons in the presence of neurotoxic materials by activating autophagic lysosome function.

Compound 1 is a poorly soluble drug that is practically insoluble in aqueous media. Therefore, Mekinist, supplied as 0.5 mg and 2 mg tablets, contains dimethyl sulfoxide (DMSO) solvate of Compound 1 as an Active Pharmaceutical Ingredient (API), which has improved solubility compared to Compound 1. As described in WO 2012/088033, however, the DMSO solvate of Compound 1 has disadvantages such as the tendency to revert to an insoluble desolvated form when exposed to moisture during the formulation process, slow dissolution of the compound from solid dosage forms, and photo-instability. In WO 2012/088033, the inventors attempted to address such concerns by using excipients substantially free of water, micronizing the drug particles, and introducing film coating. However, Mekinist® tablets manufactured as such still have the issue where the API, DMSO solvate of Compound 1, loses DMSO and reverts to the insoluble desolvated form over time, especially at high temperature and humidity. Since this adversely affects tablet dissolution, the approved storage condition of Mekinist® is refrigerated storage (2-8° C.), which is unusual for a tablet. Further, Mekinist® must be kept in a tightly closed bottle and protected from light to avoid exposure to moisture and light. Furthermore, Mekinist® should be taken at least 1 hour before or 2 hours after a meal because the absorption of the drug can be affected by food.

Accordingly, there is a need for a formulation which can be stored at room temperature, is stable upon long-term storage, and exhibits adequate dissolution profiles even in the varying conditions of the gastrointestinal tract so that the food effect can be minimized.

### SUMMARY

An objective of the present invention is to provide a stable liquid pharmaceutical formulation of Compound 1.

Another objective is to provide a liquid pharmaceutical formulation of Compound 1 which can be stored at room temperature, is stable upon long-term storage, and exhibits good oral bioavailability when taken orally.

Another objective is to provide an oral liquid pharmaceutical formulation of Compound 1 using a self-emulsifying drug delivery system (SEDDS).

While studying to solve the problems above, the inventors found that a stable liquid formulation of Compound 1 can be made by using a specific combination of solubilizers comprising an oil(s) (lipid vehicle), a water-insoluble surfactant(s) having a hydrophile-lipophile balance (HLB) of less than 11, a water-soluble surfactant(s) having an HLB of 11 or greater, and/or a hydrophilic cosolvent(s). In addition, the inventors found that a lipid liquid composition of Compound 1 exhibiting the properties of SEDDS when exposed to an aqueous medium, as well as a stable hydrophilic liquid composition of Compound 1, can be provided.

In an aspect of the present invention, a lipid liquid composition comprising Compound 1 or a pharmaceutically acceptable salt or solvate thereof and a pharmaceutically acceptable solubilizer(s) is provided. In the lipid liquid composition, the solubilizer consists of, based on the total weight of the solubilizer, about 0 to about 20 weight % of one or more oils, about 0 to about 30 weight % of one or more water-insoluble surfactants having an HLB of less than 11, about 50 to about 70 weight % of one or more water-soluble surfactants having an HLB of 11 or greater, and about 10 to about 40 weight % of one or more hydrophilic cosolvents, wherein at least one of the oil and the water-insoluble surfactant is included as an essential component. The lipid liquid composition may further contain one or more precipitation inhibitors. The precipitation inhibitor can be added so that the weight ratio of Compound 1 to the precipitation inhibitor is in the range of approximately 1:1 to approximately 1:10. Preferably, the weight ratio of Compound 1 to the precipitation inhibitor is approximately 1:4 to approximately 1:9, more preferably about 1:4. The lipid liquid composition comprises, based on the total weight of the composition, about 0.2 weight % (2 mg/g) or less of Compound 1, preferably about 0.01 weight % (0.1 mg/g) to about 0.125 weight % (1.25 mg/g), more preferably about 0.05 weight % (0.5 mg/g) to about 0.1 weight % (1 mg/g) of Compound 1.

In another aspect, the present invention provides a hydrophilic liquid composition comprising Compound 1 or a pharmaceutically acceptable salt or solvate thereof, a pharmaceutically acceptable solubilizer(s), and a precipitation inhibitor(s). In the hydrophilic liquid composition, the solubilizer consists of, based on the total weight of the solubilizer, about 3 weight % of one or more hydrophilic surfactants having an HLB of 11 or greater and about 97 weight % of one or more hydrophilic cosolvents. The hydrophilic liquid composition comprises, based on the total weight of the composition, about 0.3 weight % (3 mg/g) or less of Compound 1, preferably about 0.01 weight % (0.1 mg/g) to about 0.2 weight % (2 mg/g), more preferably about 0.05 weight % (0.5 mg/g) to about 0.15 weight % (1.5 mg/g) of Compound 1. The precipitation inhibitor can be added so that the weight ratio of Compound 1 to the precipitation inhibitor is in the range of approximately 1:1 to approximately 1:10. Preferably, the weight ratio of Compound 1 to the precipitation inhibitor is approximately 1:4 to approximately 1:9, more preferably about 1:4.

In the case the liquid composition comprises one or more oils, the suitable oil is a fatty acid ester of glycerol or a derivative thereof. Preferably, the oil is vegetable oil, medium chain triglyceride (MCT) oil, or mono-, di- or tri-glyceride of C₁₈-C₂₀ unsaturated fatty acid. More preferably, the oil may be at least one selected from the group consisting of glyceryl monooleate, medium chain triglyceride oil, soybean oil, and glyceryl monolinoleate.

In the case the liquid composition comprises one or more water-insoluble surfactant having an HLB of less than 11, the suitable water-insoluble surfactant is phospholipid, lecithin, phosphatidylcholine, sorbitan fatty acid ester, propylene glycol fatty acid ester, glycerol fatty acid ester, or polyoxylglyceride. The fatty acid included in the water-insoluble surfactant is preferably a saturated or unsaturated fatty acid having 8 to 18 carbon atoms. More preferably, the water-insoluble surfactant may be at least one selected from sorbitan monooleate, phosphatidylcholine in propylene glycol, phosphatidylcholine in medium chain triglyceride oil, lecithin, glyceryl monocaprylocaprate, propylene glycol monocaprylate, and oleoyl polyoxylglyceride.

In the liquid composition, the one or more water-soluble surfactant having HLB of 11 or greater is suitably polyoxyl castor oil, polyoxyl hydrogenated castor oil, polyoxyl sorbitan fatty acid ester, polyoxyl tocopherol ester derivatives, or polyoxylglyceride. The fatty acid included in the water-soluble surfactant is preferably a saturated or unsaturated fatty acid having 8 to 18 carbon atoms. More preferably, the water-soluble surfactant may be at least one selected from the group consisting of polyoxyl 35 castor oil, polyoxyl 40 hydrogenated castor oil, polysorbate, tocopherol polyethylene glycol succinate, and C₈-C₁₂ fatty acid polyoxylglyceride (e.g., lauroyl polyoxylglyceride or caprylocaproyl polyoxylglyceride).

In the liquid composition, the one or more hydrophilic cosolvent is suitably (poly)alkylene glycol, (poly)alkylene glycol alkyl ether, or alcohol. Preferably, the hydrophilic cosolvent may be at least one selected from the group consisting of diethylene glycol monoethyl ether, propylene glycol, polyethylene glycol, and ethanol.

In the case the liquid composition comprises one or more precipitation inhibitors, the precipitation inhibitor is suitably polymers such as polyvinylpyrrolidone or copolymers thereof, cellulose derivatives, or polyvinyl caprolactam-polyvinyl acetate-polyethylene glycol graft copolymer. Preferably, the precipitation inhibitor may be at least one selected from the group consisting of copovidone, hydroxypropyl methylcellulose (HPMC), hydroxypropyl methylcellulose acetate succinate (HPMCAS), and Soluplus.

In another aspect of the invention, the lipid liquid composition may comprise solubilizers consisting of, based on the total weight of the solubilizers, about 5 weight % of one or more oils, about 60 weight % of one or more water-soluble surfactants, and about 35 weight % of one or more hydrophilic cosolvents.

In yet another aspect of the invention, the lipid liquid composition may comprise solubilizers consisting of, based on the total weight of the solubilizers, about 30 weight % of one or more water-insoluble surfactants, about 55 weight % of one or more water-soluble surfactants, and about 15 weight % of one or more hydrophilic cosolvents.

In yet another aspect of the invention, the lipid liquid composition may comprise solubilizers consisting of, based on the total weight of the solubilizers, about 20 weight % of one or more water-insoluble surfactants, about 66 weight % of one or more water-soluble surfactants, and about 14 weight % of one or more hydrophilic cosolvents.

In yet another aspect of the invention, the lipid liquid composition may comprise solubilizers consisting of, based on the total weight of the solubilizers, about 25 weight % of one or more water-insoluble surfactants, about 60 weight % of one or more water-soluble surfactants, and about 15 weight % of one or more hydrophilic cosolvents.

In yet another aspect of the invention, the lipid liquid composition may comprise solubilizers consisting of, based on the total weight of the solubilizers, about 5 weight % of one or more oils, about 20 weight % of one or more water-insoluble surfactants, about 60 weight % of one or more water-soluble surfactants, and about 15 weight % of one or more hydrophilic cosolvents.

In yet another aspect of the invention, the liquid pharmaceutical composition may comprise one or more antioxidants to prevent oxidation of the lipid vehicles. In addition, the liquid pharmaceutical composition may comprise additional pharmaceutically acceptable carriers depending on the type of the formulation.

In yet another aspect of the invention, the liquid pharmaceutical composition can be formulated into an oral dosage form. Preferably, the liquid pharmaceutical composition can be filled into hard or soft capsules to obtain capsule formulations. The liquid pharmaceutical composition can be formulated into liquid preparations for oral administration such as oral liquids, syrups, jellies, concentrates, and the like.

The present invention can provide a liquid pharmaceutical composition of Compound 1 which has good solubility for Compound 1, can be stored at room temperature, is stable upon long term storage, and exhibits good bioavailability. In addition, the stable liquid pharmaceutical formulation of the present invention can form SEDDS and when taken orally, exhibits a good dissolution profile under the varying conditions of the gastrointestinal tract, thereby exerting minimal food effect and improved drug absorption.

### BRIEF DESCRIPTION OF THE DRAWINGS

FIG. 1 is a graph showing the solubility of Compound 1 in various solubilizers.

FIG. 2 is the result of emulsion characterization in water of the liquid active compositions according to some embodiments of the invention.

FIGS. 3A and 3B are photographs showing the appearance change upon addition of precipitation inhibitors to the solubilizer compositions according to some embodiments of the invention.

FIG. 4 is a pharmacokinetic (PK) profile graph showing the plasma concentration changes of Compound 1 upon oral administration of the liquid active compositions according to some embodiments of the invention to Beagle dogs at around 7˜8 months old.

FIGS. 5A to 5D is the results of the in-vial stability test of the liquid active composition (F5 (L1)) according to an embodiment of the invention stored under 3 different storage conditions (25° C./60% relative humidity (RH), 30° C./65% RH and 40° C./75% RH) for 4 weeks (5A), 8 weeks (5B), 12 weeks (5C), and 24 weeks (5D).

FIGS. 6A to 6D is the results of the in-vial stability test of the liquid active composition (F5_S (L1)) according to an embodiment of the invention stored under 3 different storage conditions (25° C./60% RH, 30° C./65% RH and 40° C./75% RH) for 4 weeks (6A), 8 weeks (6B), 12 weeks (6C), and 24 weeks (6D).

FIGS. 7A to 7D is the results of the in-vial stability test of the liquid active composition (F5_S (L2)) according to an embodiment of the invention stored under 3 different storage conditions (25° C./60% RH, 30° C./65% RH and 40° C./75% RH) for 4 weeks (7A), 8 weeks (7B), 12 weeks (7C), and 24 weeks (7D).

FIGS. 8A to 8D is the results of the in-vial stability test of the liquid active composition (F7 (L1)) according to an embodiment of the invention stored under 3 different storage conditions (25° C./60% RH, 30° C./65% RH and 40° C./75% RH) for 4 weeks (8A), 8 weeks (8B), 12 weeks (8C), and 24 weeks (8D).

FIGS. 9A to 9D is the results of the in-vial stability test of the liquid active composition (F5_S (L1)) according to an embodiment of the invention stored under 3 different storage conditions (25° C./60% RH, 30° C./65% RH and 40° C./75% RH) for 4 weeks (9A), 8 weeks (9B), 12 weeks (9C), and 24 weeks (9D).

FIGS. 10A to 10D is the results of the in-vial stability test of the liquid active composition (F5_S (L2)) according to an embodiment of the invention stored under 3 different storage conditions (25° C./60% RH, 30° C./65% RH and 40° C./75% RH) for 4 weeks (10A), 8 weeks (10B), 12 weeks (10C), and 24 weeks (10D).

FIG. 11 is a combined graph of the in vitro dispersion testing results in simulated gastric fluid (SGF) and in vitro lipolysis profiles of the F5 series hydrophilic liquid active compositions according to some embodiments of the invention.

FIG. 12 is a combined graph of the in vitro dispersion testing results in simulated gastric fluid and in vitro lipolysis profiles of the F7 series lipid-based liquid active compositions according to some embodiments of the invention.

### DESCRIPTION OF EXEMPLARY EMBODIMENTS

Unless otherwise defined, the technical and scientific terms used herein have the same meaning as commonly understood to those skilled in the art. Any methods and materials similar or equivalent to those described herein can be used to carry out or test the present invention.

The present invention can be modified or applied within the scope of the claims and equivalents thereto.

### The Liquid Pharmaceutical Formulation or Composition

The “liquid” pharmaceutical formulation or “liquid” pharmaceutical composition includes a solution, which is a homogeneous mixture of substances in a liquid medium, as well as a semi-solid, emulsion or suspension in which substances are dispersed in a liquid medium homogeneously or non-homogeneously.

“Solubilizer” is a term collectively referring to pharmaceutically acceptable excipients, vehicles, carriers, fillers, diluents, emulsifiers, dispersants, suspending agents, oils, solvents, cosolvents, surfactants, co-surfactants, and the like in the form of semi-solid or liquid that can be used to dissolve, disperse, or suspend a drug substance in the pharmaceutical field. The solubilizer can be used as a single agent or a mixture of two or more agents.

“Oil” refers to a lipid solvent that can be used to dissolve, disperse, or suspend a drug substance in the pharmaceutical field. The examples of the oil that can be used in the present invention include, but are not limited to, (i) fatty acid esters of glycerol, i.e., monoglycerides, diglycerides, triglycerides, medium chain triglycerides (MCT), fatty acid triglycerides, or fatty acid mono-, di-, tri-glycerides, e.g., mono- di- and/or tri-glycerides of C₁₈-C₂₀ unsaturated fatty acid, for example, glyceryl monooleate (e.g., Peceol™) or glyceryl monolinoleate (e.g., Maisine® CC), (ii) esters of fatty acid and primary alkanol, particularly esters of C₈-C₂₀ fatty acid and C₂-C₃ primary alkanol, for example, isopropyl myristate, isopropyl palmitate, ethyl linoleate, or ethyl oleate, (iii) natural vegetable or animal oils, e.g., sesame oil, peanut oil, castor oil, olive oil, corn oil, cotton seed oil, soybean oil, peppermint oil, coconut oil, palm seed oil, Safflower oil, or fish oil, (iv) carbohydrates, e.g., squalene or squalane, (v) free fatty acids, e.g., lipid oleic acid or linoleic acid, (vi) tocopherols, e.g., dl-α-tocopheryl acetate, and the like. The oil can be used as a single agent or in the form of a mixture of two or more oils.

The non-limiting examples of “surfactant” is (i) a reaction product between natural or hydrogenated vegetable oils and ethylene glycol, e.g., polyoxyl natural or hydrogenated vegetable oils, for example, polyoxyl natural or hydrogenated castor oil (e.g., Kolliphor®, Cremophor®), polyoxyl tocopherol ester derivatives (e.g., tocopherol polyethylene glycol succinate), (ii) polyoxyl-sorbitan-fatty acid esters, e.g., mono-or tri-lauryl, palmityl, stearyl, or oleyl esters (e.g., Tween™), (iii) polyoxyl fatty acid esters, e.g., polyoxyl stearic acid esters (e.g., Myrj™), (iv) polyoxyethylene-polyoxypropylene copolymer (e.g., Poloxamer, Pluronic®), (v) sodium dioctyl sulfosuccinate or sodium lauryl sulfonate, (vi) phospholipids (particularly, lecithin) or phosphatidylcholines (e.g., Phosal® 50 PG, Phosal 53 MCT), (vii) fatty acid esters of sorbitan (e.g., Span), e.g., sorbitan monooleate, sorbitan monoisostearate, sorbitan monolaurate, sorbitan monopalmitate, or sorbitan monostearate, (viii) mono- or di-fatty acid esters of propylene glycol, e.g., propylene glycol monocaprylate (e.g., Capryol® 90), propylene glycol dicaprylate, propylene glycol dilaurate, propylene glycol isostearate, propylene glycol laurate, propylene glycol ricinoleate, propylene glycol caprylic-capric acid diester (e.g., Miglyol® 840), (ix) transesterification reaction products between natural vegetable oil triglyceride and polyalkylene polyol, e.g., polyoxylglycerides, for example oleoyl polyoxylglycerides (e.g., Labrafil® M 1944CS), lauroyl polyoxylglycerides (e.g., Gelucire® 44/14), caprylocaproyl polyoxylglyceride (e.g., Labrasol® ALF), (x) fatty acid esters of glycerol, e.g., glyceryl monocaprylocaprate (e.g., Capmul® MCM), (xi) sterol or its derivatives, e.g., cholesterol, phytosterol or sitosterol, (xii) pentaerythrite-fatty acid ester, pentaerythritol fatty acid ester or polyalkylene glycol ester, e.g., pentaerythritol-dioleate, -distearate, -monolaurate, -polyethylene glycol ether, and -monostearate, and the like. These can be used as a single agent or in the form of a mixture of two or more agents.

“Water-insoluble surfactant” refers to a surfactant having an HLB value less than 11. It is a lipophilic surfactant insoluble or practically insoluble in an aqueous medium. In some instances, it may include a component(s) categorized as “oil” whose HLB is 1 or less.

“Water-soluble surfactant” refers to a surfactant having an HLB value of 11 or greater.

It refers to a hydrophilic excipient that is completely miscible or has a high solubility or dispersibility in an aqueous medium.

“Cosolvent” refers to any solvent that can assist in solubilizing an active pharmaceutical ingredient. Non-limiting examples of “hydrophilic cosolvent” are nonionic polymers having a (poly)oxyalkylene moiety, for example, (poly)alkylene glycol (e.g., polyethylene glycol such as PEG200, PEG400, PEG600, etc., propylene glycol), (poly)alkylene glycol alkyl ether (e.g., diethylene glycol monoalkyl ether (e.g., Transcutol® HP)), alcohol (e.g., ethanol) or a mixture thereof.

“Precipitation inhibitor” refers to a polymeric excipient that mediates supersaturation stabilization and/or inhibits and/or retards (rapid) precipitation or aggregation in a liquid composition or gastrointestinal tract. Examples of such a precipitation inhibitor include, but are not limited to, starch; cellulose and its derivatives such as microcrystalline cellulose, carboxymethylcellulose, hydroxypropyl methyl cellulose (HPMC) (e.g., METHOCEL™ E5LV), HPMC acetate succinate (HPMCAS) (e.g., HPMCAS-H, HPMCAS-M, Shin-Etsu AQOAT®), HPMC phthalate (HPMCP) (e.g., HPMCP-HP55, HPMCP-HP55S), hydroxypropyl cellulose, hydroxyethyl cellulose, ethyl cellulose; sugar such as lactose, sucrose, dextrose, glucose, maltodextrin, mannitol, xylitol; polymethacrylate (e.g., Eudragit®, such as Eudragit L100-55), polyvinylpyrrolidone or copolymers thereof (e.g., polyvinylpyrrolidone vinyl acetate copolymer (PVP/VA), copovidone (e.g., Kollidon® VA64), or other copolymers (e.g., polyvinyl caprolactam-polyvinyl acetate-polyethylene glycol graft copolymer (such as Soluplus®). These can be used as a single agent or in the form of a mixture of two or more agents.

“About” or “approximately” is used to cover numerical values in the range of +10% of the value referred to in the specification of the present invention.

In an embodiment of the present invention, the oil is at least one selected from the group consisting of glyceryl monooleate (e.g., Peceol), medium chain triglyceride oil, soybean oil, and glyceryl monolinoleate (e.g., Maisine CC). Preferably, the oil is glyceryl monooleate.

The lipid liquid composition of the present invention comprises an oil(s) preferably in the amount of approximately 20 weight % or less, because it can solubilize Compound 1 by easily forming an emulsion in in vivo environment such as the gastrointestinal tract.

It is more preferable for the composition to comprise the oil(s) in an amount of about 5 weight % or less. In the case the lipid liquid composition contains a water-insoluble surfactant(s), oils may not be included in the composition.

In an embodiment of the present invention, the water-insoluble surfactant is at least one selected from the group consisting of sorbitan monooleate (e.g., Span), phosphatidylcholine in propylene glycol (e.g., Phosal 53 MCT), phosphatidylcholine in medium chain triglyceride oil (e.g., Phosal 50 PG), lecithin, glyceryl monocaprylocaprate (e.g., Capmul MCM), propylene glycol monocaprylate (e.g., Capryol 90), and oleoyl polyoxylglyceride (e.g., Labrafil M 1944CS). Preferably, the water-insoluble surfactant is monocaprylocaprate.

In the lipid liquid composition, the water-insoluble surfactant is preferably in the amount of approximately 30 weight % or less. When the lipid liquid composition comprises an oil(s), the water-insoluble surfactant may not be included.

In an embodiment of the present invention, the water-soluble surfactant is at least one selected from the group consisting of fatty acid polyoxyl glyceride (e.g., Gelucire 44/14, Labrasol ALF), polyoxyl 35 castor oil (e.g., Kolliphor EL, previously known as Cremophor EL), polyoxyl 40 hydrogenated castor oil (e.g., Kolliphor RH 40, previously known as Cremophor RH 40), tocopherol polyethylene glycol succinate (e.g., Vitamin E TPGS) and polysorbate (e.g., Tween). Preferably, the water-soluble surfactant is fatty acid polyoxyl glyceride or polysorbate.

In the lipid liquid composition, the water-soluble surfactant is preferably in the amount of about 50 weight % to about 70 weight %. If the water-soluble surfactant is included in the amount of less than 50 weight % or more than 70 weight %, the optimal solubility of Compound 1 in the composition is not achieved. The water-soluble surfactant is more preferably in the amount of about 55 weight % to about 66 weight %.

In an embodiment of the present invention, the hydrophilic cosolvent is at least one selected from the group consisting of diethylene glycol monoethyl ether (e.g., Transcutol HP), propylene glycol, polyethylene glycol (e.g., PEG200 to PEG600), and alcohol. Preferably, the hydrophilic cosolvent is diethylene glycol monoethyl ether or polyethylene glycol.

In the lipid liquid composition, the hydrophilic cosolvent is preferably in the amount of about 10 weight % to about 40 weight %. If the hydrophilic cosolvent is included in the amount of less than 10 weight % or more than 40 weight %, the optimal solubility of Compound 1 in the composition is not achieved. The hydrophilic cosolvent is more preferably in the amount of about 15 weight % to about 35 weight %.

In the lipid liquid composition of the present invention, the solubilizers may be comprised of the following components (the weight % is based on the total weight of the solubilizers):
(a) about 5 weight % of one or more oils, about 60 weight % of one or more water-soluble surfactants, and about 35 weight % of one or more cosolvents;
(b) about 30 weight % of one or more water-insoluble surfactants, about 55 weight % of one or more water-soluble surfactants, and about 15 weight % of one or more hydrophilic cosolvents;
(c) about 20 weight % of one or more water-insoluble surfactants, about 66 weight % of one or more water-soluble surfactants, and about 14 weight % of one or more hydrophilic cosolvents;
(d) about 25 weight % of one or more water-insoluble surfactants, about 60 weight % of one or more water-soluble surfactants, and about 15 weight % of one or more hydrophilic cosolvents; or
(e) about 5 weight % of one or more oils, about 20 weight % of one or more water-insoluble surfactants, about 60 weight % of one or more water-soluble surfactants, and about 15 weight % of one or more hydrophilic cosolvents.

In an embodiment, in the case the liquid composition comprises a precipitation inhibitor(s), the precipitation inhibitor may be at least one selected from the group consisting of polyvinyl caprolactam-polyvinyl acetate-polyethylene glycol graft copolymer (e.g., Soluplus), copovidone (e.g., Kollidon VA64), hydroxypropyl methylcellulose (HPMC, e.g., METHOCEL™ E5LV), and hydroxypropyl methylcellulose acetate succinate (HPMCAS, e.g., Shin-Etsu AQOAT). Preferably, the precipitation inhibitor is Soluplus or copovidone. More preferably, the precipitation inhibitor is Soluplus. In the liquid composition, the precipitation inhibitor is used to prevent precipitation of Compound 1 in the gastrointestinal tract environment and can be used in various amounts. For example, the precipitation inhibitor can be added so that the weight ratio of Compound 1 to the precipitation inhibitor is in the range of approximately 1:1 to approximately 1:10. Preferably, the weight ratio of Compound 1 to the precipitation inhibitor is approximately 1:4 to approximately 1:9. More preferably, the weight ratio of Compound 1 to the precipitation inhibitor is about 1:4.

In a preferred embodiment, the lipid liquid composition comprises a drug, which is Compound 1 or a pharmaceutically acceptable salt or solvate thereof, and pharmaceutically acceptable solubilizers, wherein the solubilizers consist of, based on the total weight of the solubilizers, about 5 weight % of Peceol, about 35 weight % of PEG400, about 20 weight % of Labrasol ALF, and about 40 weight % of Gelucire 44/14, wherein the drug is contained in an amount of about 0.01 weight % to about 0.2 weight %, about 0.01 weight % to about 0.125 weight %, or about 0.05 weight % to about 0.1 weight %, based on the total weight of the composition. The lipid liquid composition may additionally comprise Soluplus as a precipitation inhibitor in the amount of approximately 4 times the amount of Compound 1.

In a preferred embodiment, the hydrophilic liquid composition comprises a drug, which is Compound 1 or a pharmaceutically acceptable salt or solvate thereof, pharmaceutically acceptable solubilizers, and a precipitation inhibitor, wherein the solubilizers consist of, based on the total weight of the solubilizers, about 77 weight % of PEG400, about 20 weight % of Transcutol HP, and about 3 weight % of Tween 80, wherein the drug is contained in an amount of about 0.01 weight % to about 0.3 weight %, about 0.01 weight % to about 0.2 weight %, or about 0.05 weight % to about 0.15 weight %, based on the total weight of the composition. The precipitation inhibitor may be Soluplus in the amount of approximately 4 times the amount of Compound 1.

The lipid pharmaceutical formulation or composition may additionally comprise one or more antioxidants to prevent oxidation of lipid excipients. Examples of such an antioxidant include, but are not limited to, chelating agent, reactive oxygen scavenger, and chain terminating agent, for example, ethylene diamine tetraacetic acid (EDTA), citric acid, ascorbic acid, butylated hydroxytoluene (BHT), butylated hydroxyanisole (BHA), sodium sulfite, p-aminobenzoic acid, glutathione, propyl gallate, cysteine, methionine, ethanol, and N-acetylcysteine. Preferably, the antioxidant is butylated hydroxytoluene.

The liquid pharmaceutical formulation or composition can be prepared with a method commonly used in the pharmaceutical field. In some embodiments, the liquid pharmaceutical formulation or composition can be prepared by melting any semisolid solubilizers, mixing solubilizers to form a mixture of the solubilizers, then adding precipitation inhibitors and/or antioxidants to the mixture, if needed, and adding an amount of Compound 1 to obtain an active composition.

The drug, which is included in the liquid pharmaceutical formulation or composition as an active pharmaceutical ingredient, may be Compound 1 in the form of a free base or a pharmaceutically acceptable salt or solvate thereof and is not specifically limited. Preferably, the liquid pharmaceutical formulation or composition comprises Compound 1 in the form of a free base. However, salts or solvates (e.g., DMSO or acetic acid solvate) of Compound 1 may also be used, insofar as the solubility to the other ingredients is not remarkably lower than that of the free base form.

The solvate of Compound 1 that can be used in the preparation of the liquid pharmaceutical formulation or composition includes, but is not limited to, hydrate, or other solvates such as dimethyl sulfoxide, acetic acid, ethanol, nitromethane, chlorobenzene, 1-pentanol, isopropyl alcohol, ethylene glycol, and 3-methyl-1-butanol solvate, and the like. Any solvate that can be formed with a pharmaceutically acceptable solvent can be included.

The salt of Compound 1 that can be used in the preparation of the liquid pharmaceutical formulation or composition includes any pharmaceutically acceptable salts formed from inorganic acids or organic acids.

### Stability of the Liquid Pharmaceutical Formulation or Composition

The “stable” liquid pharmaceutical formulation or composition means that Compound 1 is not precipitated, aggregated or crystallized in the formulation or composition, or that the formulation or composition is maintained in the state where such precipitation, aggregation or crystallization is minimized. The stability of the liquid pharmaceutical formulation or composition can be evaluated by measuring the recovery (%) of Compound 1 with a chromatography technique such as HPLC or the total impurities (%) in the composition or formulation stored for a certain period of time at a certain temperature and humidity. In some embodiments, the storage period for the safety test is at least 14 days, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or longer. In some embodiments, the storage temperature for the safety test is any temperature between about 0° C. and about 70° C., for example, about 0° C., about 4° C.-8° C., about 15° C., about 20° C., about 25° C., about 30° C., about 35° C., about 37° C., about 40° C., or about 45° C. In some embodiments, the storage humidity for the safety test is any humidity between about 50% RH and about 80% RH, for example, about 50% RH, about 60% RH, about 65% RH, about 70% RH, about 75% RH, or about 80% RH. “About” used for describing the temperatures covers ±2° C. of the indicated temperature. “About” used for describing the humidity covers±5% RH of the indicated humidity.

In an embodiment, the liquid pharmaceutical formulation or composition is regarded stable if the % recovery of Compound 1 after storage at 25° C.±2° C. and 60% RH±5% RH (room temperature condition) for 2 weeks, 4 weeks, 8 weeks, 12 weeks, or 24 weeks is at least about 95%, at least about 96%, at least about 97%, or at least about 98%.

In an embodiment, the liquid pharmaceutical formulation or composition is regarded stable if the % recovery of Compound 1 after storage at 30° C.±2° C. and 65% RH±5% RH (intermediate condition) for 2 weeks, 4 weeks, 8 weeks, 12 weeks, or 24 weeks is at least about 90%, at least about 95%, at least about 96%, at least about 97%, or at least about 98%.

In an embodiment, the liquid pharmaceutical formulation or composition is regarded stable if the % recovery of Compound 1 after storage at 40° C.±2° C. and 75% RH±5% (accelerated condition) for 2 weeks, 4 weeks, 8 weeks, 12 weeks, or 24 weeks is at least about 85%, at least about 90%, at least about 95%, at least about 96%, at least about 97%, or at least about 98%.

In an embodiment, the liquid pharmaceutical formulation or composition is regarded stable if the total impurities (%) after storage at 25° C.±2° C. and 60% RH±5% RH (room temperature condition) for 2 weeks, 4 weeks, 8 weeks, 12 weeks, or 24 weeks is about 2% or less, about 1% or less, about 0.5% or less, about 0.1% or less, or about 0.05% or less.

In an embodiment, the liquid pharmaceutical formulation or composition is regarded stable if the total impurities (%) after storage at 30° C.±2° C. and 65% RH±5% RH (intermediate condition) for 2 weeks, 4 weeks, 8 weeks, 12 weeks, or 24 weeks is about 2% or less, about 1% or less, about 0.5% or less, about 0.1% or less, or about 0.05% or less.

In an embodiment, the liquid pharmaceutical formulation or composition is regarded stable if the total impurities (%) after storage at 40° C.±2° C. and 75% RH±5% RH (accelerated condition) for 2 weeks, 4 weeks, 8 weeks, 12 weeks, or 24 weeks is about 2% or less, about 1% or less, about 0.5% or less, about 0.1% or less, or about 0.05% or less.

In a preferred embodiment, the stable liquid pharmaceutical formulation or composition exhibits, even after storage at the accelerated condition for 12 weeks to 24 weeks, % recovery of Compound 1 within 95.0-105.0% label claim, which is the acceptance criteria for a medicinal product.

In a preferred embodiment, the stable liquid pharmaceutical formulation or composition exhibits, even after storage at the accelerated condition for 12 weeks to 24 weeks, total impurities of 2.0% or less and individual impurities less than the Identification Threshold (0.5%) of ICH (The International Council for Harmonisation).

### Self-Emulsifying Drug Delivery System (SEDDS)

SEDDS is one of the formulation strategies that can be used to enhance the absorption of a poorly soluble drug, resulting in an improvement in bioavailability. SEDDS, which is an isotropic mixture of oils (lipids), surfactants, and co-surfactants (cosolvents), can be immediately dispersed in an aqueous media upon dilution and gentle agitation to form a homogeneous emulsion.

The lipid liquid composition comprising a solubilizer mixture consisting of oil/water-insoluble surfactant/water-soluble surfactant/hydrophilic cosolvent is considered to form SEDDS in the gastric and/or intestinal environment of the body. Thus, when the lipid liquid composition is administered to a human body, it is rapidly self-emulsified in the gastrointestinal tract to form SEDDS, resulting in the absorption of Compound 1 into the body within several minutes and thereby maximizing the bioavailability due to enhanced absorption.

The SEDDS described herein may comprise liquid droplets in size (i.e., diameter) ranging from about 5 nm to about 10 μm. For example, in an embodiment, the droplet size is in the range of about 500 nm to about 4 μm, about 5 nm to about 1 μm, about 50 nm to about 750 nm, about 50 nm to about 200 nm, about 100 nm to about 500 nm, or about 150 nm to about 350 nm.

Formulation into a Dosage Form

The liquid pharmaceutical formulation or composition described herein can be formulated into oral dosage forms with a method commonly used in the pharmaceutical field. The oral dosage forms may be a solid form such as capsules with the liquid pharmaceutical formulation or composition of the present invention contained inside a soft or hard capsule shell, or a liquid form such as a liquid for oral administration (e.g., elixirs, suspensions, emulsions, lemonades), syrups, oral jellies, or oral concentrates that are diluted before administration with a suitable diluent such as water. For example, the liquid pharmaceutical formulation or composition is formulated into a capsule. Preferably, the liquid pharmaceutical formulation or composition may be formulated to contain Compound 1 in an amount of about 2.0 mg, about 1.5 mg, about 1.25 mg, about 1.0 mg, about 0.75 mg, about 0.5 mg, about 0.25 mg, about 0.1 mg, or about 0.05 mg per a capsule. The liquid pharmaceutical formulation or composition may be formulated into liquid preparations for oral administration containing Compound 1 at a concentration of about 2.0 mg/ml, about 1.5 mg/ml, about 1.25 mg/ml, about 1.0 mg/ml, about 0.75 mg/ml, about 0.5 mg/ml, about 0.25 mg/ml, about 0.1 mg/ml, about 0.05 mg/ml, about 0.025 mg/ml, about 0.01 mg/ml, or about 0.005 mg/ml. Preferably, the liquid pharmaceutical formulation or composition may be formulated to contain Compound 1 in an amount of about 2.0 mg, about 1.5 mg, about 1.25 mg, about 1.0 mg, about 0.75 mg, about 0.5 mg, about 0.25 mg, about 0.1 mg, or about 0.05 mg per 1 to 50 ml of a liquid preparation for oral administration. For this purpose, the liquid pharmaceutical formulation or composition may further comprise pharmaceutically acceptable additives such as viscosity controlling agents, flavoring agents, preservatives, coloring agents, sweetening agents, gelatin, diluents, and the like that can be commonly selected in the pharmaceutical field.

The dose can be varied depending on the patient's body weight, age, sex, health condition, administration time, method, period or interval, severity of the disease, and the like. For example, the lipid pharmaceutical formulation or composition can be administered once a day at a dose of about 2.0 mg, about 1.5 mg, about 1.25 mg, about 1.0 mg, about 0.75 mg, about 0.5 mg, about 0.25 mg, about 0.1 mg, or about 0.05 mg.

The pharmaceutical composition may additionally comprise a pharmacologically active agent(s) other than the compound described herein. The pharmacological activity of the other active agent may be the same or different from the pharmacological activity of the compound described herein.

### EXAMPLES

The following examples describe the present invention in more detail. The examples are put forth to describe the present invention more specifically and are not intended to limit the scope of the present invention to the examples. It is evident to those skilled in the art that the examples can be modified in various ways within the scope of the claims.

Unless indicated otherwise, any numerical values used herein should be understood to cover the values within ±10% margin of error for the indicated value. All the solubilizers, excipients, and vehicles used herein are standard pharmaceutical grade. The temperatures are in degrees Celsius.

### Example 1: Preparation of the Liquid Composition

### Example 1-1. Solubility Screening and Solubilization Study

To develop a stable liquid formulation of Compound 1, the solubility of Compound 1 was measured in various solubilizers including oil/lipid vehicles, water-insoluble surfactants (HLB<11), water-soluble surfactants (HLB≥11), and hydrophilic cosolvents. The solubilizers used are described in Table 1. An excess of Compound 1 (approximately 20 mg) was added to a 2 mL Eppendorf vial containing 500 mg of a solubilizer (liquid or molten) and dissolved. After 24 hours, samples were centrifuged for 15 minutes to separate undissolved and dissolved parts. Aliquots of supernatant were taken, diluted with sample solvent, and assayed for the content of Compound 1 with HPLC.

TABLE 1

| Role | Solubilizer | Chemical description | HLB |
|---|---|---|---|
| Oil/Lipid | Peceol ™ | Glyceryl monooleate (Type | 1 |
| vehicle | | 40) | |
| | Witarix MCT oil | Medium chain triglyceride | 1 |
| | 60/40 | | |
| | Soybean oil | — | 1 |
| | Maisine ® CC | Glyceryl monolinoleate | 1 |
| Water- | Span ™ 80 | Sorbitan monooleate | 4 |
| insoluble | Phosal ® 50 PG | Phosphatidylcholine or | |
| surfactant | | lecithin in | 5 |
| | | propylene glycol | |
| (HLB < 11) | Phosal ® 53 MCT | Phosphatidylcholine or | 5 |
| | | lecithin in MCT | |
| | | (medium chain | |
| | | triglyceride) oil | |
| | Capryol ® 90 | Propylene glycol monocaprylate | 5 |
| | | (Type II) | |
| | Capmul ® MCM | Glyceryl monocaprylocaprate | 6 |
| | | (Type I) | |
| | Labrafil ® M1944CS | Oleoyl polyoxyl-6 glyceride | 9 |
| Water-soluble | Gelucire ® 44/14 | Lauroyl (C₁₂) polyoxyl-32- | 11 |
| surfactant | | glyceride | |
| (HLB ≥ 11) | Labrasol ® ALF | Caprylo (C₈) caproyl (C₁₀) | 12 |
| | | polyoxyl-8-glyceride | |
| | Kolliphor ® EL | Polyoxyl 35 castor oil | 12-14 |
| | Kolliphor ® RH 40 | Polyoxyl 40 hydrogenated castor | 14-16 |
| | | oil | |
| | Vitamin E TPGS | Tocopherol polyethylene glycol | 13 |
| | | succinate | |
| | Tween ™ 80-LQ-CQ | Polysorbate | 15 |
| Cosolvent | Transcutol ® HP | Diethylene glycol monoethyl | — |
| | | ether | |
| | propylene glycol | — | — |
| | PEG400 | Polyethylene glycol 400 | — |
| | Ethanol, anhydrous | — | — |

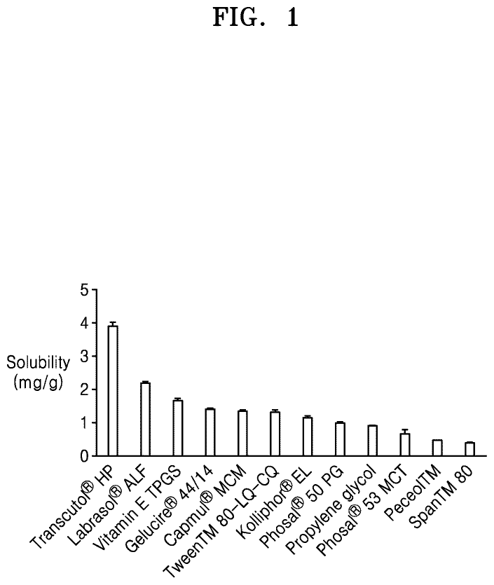

FIG. 1 shows the result of the solubility analysis of Compound 1 in the above solubilizers. In terms of the functional groups of the solubilizers, Compound 1 had a higher solubility in the medium chain (at least Ca) fatty acid derivatives and the solubilizers having (poly-) ethoxy groups.

### Example 1-2. Preparation of Active Compositions

Based on the solubilization study results, representative solubilizers from the groups of oil/water-insoluble surfactants/water-soluble surfactants/hydrophilic cosolvents were selected and composed the solubilizer mixtures as in Table 2 below.

TABLE 2

| Solubilizer | Ingredient* | F2 | F7 | F8 | F9 | F12 | F14 | F15 | F5** |
|---|---|---|---|---|---|---|---|---|---|
| Oil | Peceol | | 5 | — | 10 | — | — | 5 | — |
| | MCT oil | 23 | — | — | 12 | — | — | — | — |
| Water-insoluble | Capmul MCM | — | — | 30 | 30 | 20 | 25 | 20 | — |
| surfactant | | | | | | | | | |
| Water-soluble | Gelucire 44/14 | — | 40 | — | — | — | — | — | — |
| surfactant | Labrasol ALF | — | 20 | 55 | 48 | 40 | 50 | 50 | — |
| | Kolliphor EL | | — | — | — | 26 | 10 | 10 | — |
| | Tween 80 | 42 | — | — | — | — | — | — | 3 |
| | Vitamin E TPGS | 35 | — | — | — | — | — | — | — |
| | Transcutol HP | — | — | 15 | — | 14 | 15 | 15 | 20 |
| Hydrophilic | PEG400 | — | 35 | — | — | — | — | — | 77 |
| cosolvent | | | | | | | | | |

*The content of each ingredient is based on weight %.
**F5 is a hydrophilic composition.

Using the above solubilizer mixtures, the active compositions containing Compound 1 at 2 mg/g or 3 mg/g were prepared. The above solubilizers were mixed using a vortex at room temperature. In the case where the solubilizer mixture contained semisolid solubilizers (Peceol, Gelucire, Capmul, etc.), the semisolid solubilizers were first melted at 55° C. and then mixed. Compound 1 was added to the mixture to prepare a liquid active composition. The active composition so prepared was placed in a shaker incubator at 55° C. for 1˜2 days until complete dissolution. The appearance was observed at day 2. The samples were centrifuged for 15 minutes to check whether crystals or precipitates formed.

Table 3 shows the results of the study with the active compositions.

TABLE 3

| Active | Observation after 2 days at 55° C./900 rpm | |
|---|---|---|
| composition | 2 mg/g | 3 mg/g |
| F2* | Turbid liquid, undissolved | Large amount of |
| | Compound | undissolved |
| | 1 was observed prior to | Compound 1 |
| | centrifugation | |
| F5 | Clear liquid | Clear liquid |
| F7* | Clear liquid | Large amount of |
| | | undissolved |
| | | Compound 1 |
| F8 | Clear liquid | Large amount of |
| | | undissolved |
| | | Compound 1 |
| F9 | Turbid liquid, undissolved | Large amount of |
| | Compound | undissolved |
| | 1 after centrifugation | Compound 1 |
| F12 | Turbid liquid, but Compound 1 | Large amount of |
| | fully dissolved after | undissolved |
| | centrifugation | Compound 1 |
| F14 | Clear liquid | Large amount of |
| | | undissolved |
| | | Compound 1 |
| F15 | Clear liquid | Large amount of |
| | | undissolved |
| | | Compound 1 |

*Semisolid formulation at room temperature

The results in Table 3 show that the solubility of Compound 1 was below 2 mg/g when the amount of lipid oils is above 20% or the composition does not contain a hydrophilic cosolvent (F2 and F9). Furthermore, when the amount of the water-soluble surfactants is less than 50% (F9) or more than 70% (F2), the solubility of Compound 1 was below 2 mg/g. Meanwhile, the hydrophilic composition F5 was in a clear liquid state even at 3 mg/g of Compound 1, having good solubility of Compound 1.

### Example 2: Study on Emulsion Characterization in Water

### Example 2-1. Emulsion Stability in Water

The following compositions were prepared: F5(O) (a hydrophilic composition containing 3 mg/g Compound 1), F7(O), F8(O), F12(O), F14(O), and F15(O) (lipid compositions containing 2 mg/g Compound 1). 100 mg of each composition was dispensed into 10 ml water. The sample was mixed and centrifuged for 15 minutes at 14,000 rpm and 37° C., and then observed for precipitation.

F5(O) formed transparent emulsion and F7(O) formed translucent emulsion in water. However, F5(O) was not stable since Compound 1 precipitate was observed after 1 hour. F12(O) formed slightly turbid emulsion, while F8(O), F14(O), and F15(O) formed turbid emulsion.

### Example 2-2. Emulsion Characterization in Water

Emulsion characterization was performed for F5(O), F7(O), F12(O), and F14(O) using USP dissolution apparatus type 2. 300 μl of each composition was added to 300 ml water at 37.0±0.5° C. A standard stainless dissolution paddle rotating at 50 rpm provided gentle agitation during testing. The emulsion performance of the formulation was visually assessed according to the criteria in Table 4 below. Dynamic Light Scattering (DLS) was performed using Zetasizer Nano ZS (Malvern Instrument) at a scattering angle of 173°, 633 nm wavelength, and 37° C. to determine the droplet size of the formed emulsion. Measurements were performed in triplicates and z-average diameter was reported as the droplet size.

TABLE 4

| | Emulsification | |
|---|---|---|
| | time | |
| Dispersibility | (min) | Grade |
| Formulation spreads rapidly in water forming | <1 | A |
| clear and transparent dispersion | | |
| Formulation spreads gradually in water forming | 1-2 | B |
| translucent-bluish dispersion | | |
| Formulation droplets spread in water to form | 2-5 | C |
| turbid dispersion | | |
| Formulation shows slow and whitish dispersion | >5 | D |
| Formulation exhibits poor emulsification with | No emulsion | E |
| coalescence of oil droplets | | |

A: rapid microemulsion
B: microemulsion
C: emulsion
D: poor emulsion
E: no emulsion

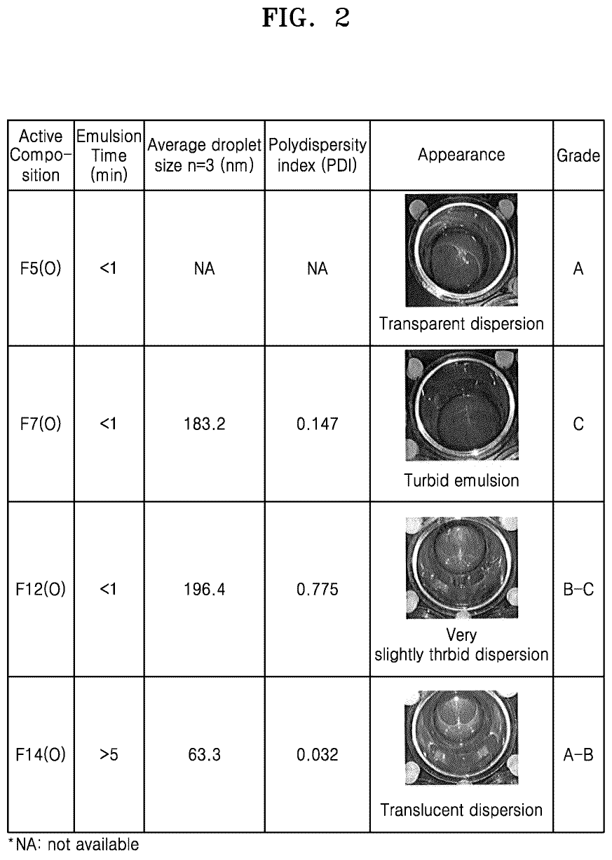

The results are shown in FIG. 2. As shown in FIG. 2, the lipid compositions F7(O), F12(O), and F14(O) formed stable emulsion in water. Among these, F14(O) had the smallest droplet size (63.3 nm) and PDI (0.032), followed by F7(O) and F12(O). These compositions are considered to function as self-emulsifying drug delivery system (SEDDS), advantageous for in vivo absorption and release of Compound 1.

Meanwhile, the hydrophilic composition F5(O) also had good emulsion properties in water with an emulsion time of less than 1 minute and formation of a transparent dispersion.

### Example 3. Emulsion Stability of Compositions with and without Precipitation Inhibitor in Water

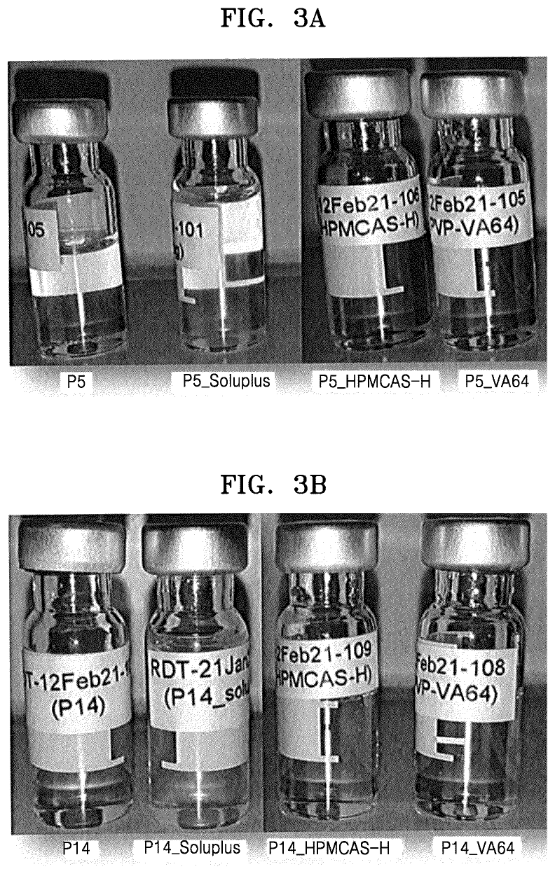

To improve emulsion stability in water and prevent precipitation of Compound 1, testing was conducted with the addition of a precipitation inhibitor (PI). As a PI agent, Soluplus® (polyvinyl caprolactam-polyvinyl acetate-polyethylene glycol graft copolymer, BASF), Kollidon® VA64 (copovidone, BASF), and Shin-Etsu AQOAT® (hydroxypropylmethylcellulose acetate succinate (HPMCAS), DuPont) were used. The ratio of the PI agent to Compound 1 was set to 4:1. The PI agent was added to the solubilizer mixtures P5 and P14, which correspond to F5 and F14 without Compound 1, respectively. The appearance was visually assessed. As shown in FIGS. 3A and 3B, the addition of HPMCAS and Kollidon VA64 to both formulations did not produce a visual change when compared to the corresponding PI agent-free control compositions. In the case of the compositions where Soluplus was added, a well-dispersed colloid with a turbid-like appearance was observed.

Next, the above PI agents were added to active compositions containing Compound 1, and emulsion stability in water was assessed with the method described in Example 2-1. Soluplus was added to F5(O) and F7(O), and VA64 was added to F14(O). All these compositions, upon being dispersed in water, were remarkably more stable than the compositions without the PI agent. F5(O) and F7(O) with Soluplus did not show any precipitate of Compound 1 and formed a stable emulsion.

### Example 4: In-Vial Stability Study

### Example 4-1. Preparation of Active Compositions in Vial

In-vial stability study was performed on compositions F5(O), F7(O), and F14(O) containing the PI agent. The ingredients of the test active compositions are described in Table 5 to 7 below.

TABLE 5

| Ingredients | F5_S(O) |
|---|---|
| Polyethylene glycol (PEG400) (weight %) | 75.56 |
| Diethylene glycol monoethyl ether (Transcutol HP) | 19.93 |
| (weight %) | |
| Polysorbate (Tween 80-LQ-CQ) (weight %) | 2.99 |
| Polyvinyl caprolactam-polyvinyl acetate-polyethylene | 1.20 |
| glycol graft copolymer (Soluplus) (weight %) | |
| Butylated hydroxytoluene (weight %) | 0.02 |
| Compound 1 (weight %) | 0.30 |
| Compound 1 (mg/g) | 3.0 |

TABLE 6

| Ingredients | F7_S(O) |
|---|---|
| Polyethylene glycol (PEG400) (weight %) | 34.64 |
| Glyceryl monooleate (Peceol) (weight %) | 4.95 |
| Caprylocaproyl polyoxylglyceride (Labrasol ALF) (weight %) | 19.80 |
| Lauroyl polyoxylglyceride (Gelucire 44/14) (weight %) | 39.59 |
| Polyvinyl caprolactam-polyvinyl acetate-polyethylene | 0.80 |
| glycol graft copolymer (Soluplus) (weight %) | |
| Butylated hydroxytoluene (weight %) | 0.02 |
| Compound 1 (weight %) | 0.20 |
| Compound 1 (mg/g) | 2.0 |

TABLE 7

| Ingredients | F14_VA64(O) |
|---|---|
| Diethylene glycol monoethyl ether (Transcutol HP) | 14.85 |
| (weight %) | |
| Caprylocaproyl polyoxylglyceride (Labrasol ALF) | 49.49 |
| (weight %) | |
| Glyceryl monocaprylocaprate (Capmul MCM) | 24.74 |
| (weight %) | |
| Polyoxyl 35 castor oil (Kolliphor EL) (weight %) | 9.90 |
| Copovidone (Kollidon VA64) (weight %) | 0.80 |
| Butylated hydroxytoluene (weight %) | 0.02 |
| Compound 1 (weight %) | 0.20 |
| Compound 1 (mg/g) | 2.0 |

The preparation method was as follows: The solubilizers were weighed and mixed together in a beaker. The compositional ratio of the solubilizers in each composition was as described in Example 1-2. Semi-solid solubilizers were melted at 50° C. before mixing. PI agent was added to the mixture. When the mixture was visually homogeneous, butylated hydroxytoluene (BHT) was added to prevent oxidation of the lipid excipients and homogenized in a shaker-incubator at 50° C. and 750 rpm. Compound 1 was weighed and added to an Erlenmeyer flask. The mixture was added on top of Compound 1 to prepare the active composition. The active composition was purged with nitrogen. The flask was covered with aluminum foil. The active composition was placed under incubation at 60° C. and 750 rpm until all Compound 1 was fully dissolved. 1.5 mL glass vials were filled with the composition, and the vial's headspace was purged with nitrogen and sealed with a polytetrafluoroethylene (PTFE) bottom coated aluminum cap.

### Example 4-2. Stability of the Composition in the Glass Vial

The glass vials containing the compositions prepared in Example 4-1 were stored for 2 weeks under the two conditions below, and stability study was performed.

$$25 °C \pm 2 °C and 60 \% RH \pm 5 \% RH$$

$$40 °C \pm 2 °C and 75 \% RH \pm 5 \% RH$$

The appearance (color, texture, appearance of precipitate, etc.) of the composition in the glass vials before the test (t=0) and after 2 weeks (t=2w) was compared with the control composition (the same composition as the test sample with the exception of not containing Compound 1). As summarized in Tables 8 to 10 below, all the compositions were stable for 2 weeks without appearance of precipitate or color change.

TABLE 8

| | | t = 2 w | t = 2 w |
|---|---|---|---|
| Composition | t = 0 | (25° C./60% RH) | (40° C./75% RH) |
| F5_S(O) | Transparent liquid | Transparent liquid | Transparent liquid |
| P5_S_control | Transparent liquid | Transparent liquid | Transparent liquid |

TABLE 9

| | | t = 2 w | t = 2 w |
|---|---|---|---|
| Composition | t = 0 | (25° C./60% RH) | (40° C./75% RH) |
| F7_S(O) | Semi-solid, | Semi-solid, white, | Liquid¹ |
| | white, opaque | wax-like beads | |
| P7_S_Control | Semi-solid, | Semi-solid, white | Transparent liquid² |
| | white, opaque | | |

^{1,2}Slowly transforms into white semi-solid including wax-like beads after 12 hours at room temperature.

TABLE 10

| | | t = 2 w | t = 2 w |
|---|---|---|---|
| | | (25° C./ | (40° C./ |
| Composition | t = 0 | 60% RH) | 75% RH) |
| F14_VA64(O) | Transparent | Transparent | Transparent |
| | liquid | liquid | liquid |
| P14_VA64_Control | Transparent | Transparent | Transparent |
| | liquid | liquid | liquid |

Aliquots of the compositions in the glass vial were taken, and the content of Compound 1 and total impurities were measured with HPLC. Measurements were performed in duplicates, and the average value was reported. Impurities were quantified by percentage area relative to the main Compound 1 peak and based on Relative Response Factor (RRF) of 1.0. As a positive control, the dried powder of Compound 1 was used and tested in the same way. Table 11 describes the parameters for the HPLC method.

TABLE 11

| Column | Thermo Hypersil Gold C18, 1.9 μm, 2.1 × 50 mm | | |
|---|---|---|---|
| Mobile Phase A | 0.1% Formic Acid (FA) in H₂O (v/v) | | |
| Mobile Phase B | 0.1% Formic Acid (FA) in ACN (v/v) | | |
| Diluent | 3:1 ACN:H₂O (v/v) | | |
| Program Type | Gradient | | |
| Gradient | Time | % Mobile Phase | % Mobile Phase |
| program | (min) | A | B |
| | 0.0 | 70 | 30 |
| | 1.0 | 70 | 30 |
| | 8.0 | 0 | 100 |
| | 10.0 | 0 | 100 |
| | 11.0 | 70 | 30 |
| | 15.0 | 70 | 30 |

| | |
|---|---|
| Flow Rate | 0.3 mL/min |
| Injection Volume | 2 μL |
| Detection Method | UV |
| Detection Wavelength | 250 nm |

The results are shown in Tables 12 and 13 below.

TABLE 12

Compound 1 Content (%)

| | t = 0 | t = 2w (40° C./75% RH) |
|---|---|---|
| F5_S(O) | 101.4 | 97.0 |
| F7_S(O) | 100.5 | 93.4 |
| F14_VA64(O) | 100.4 | 94.9 |
| Compound 1 dry powder | 100.5 | 100.3 |

TABLE 13

Total Impurities (%)

| | t = 0 | t = 2 w (40° C./75% RH) |
|---|---|---|
| F5_S(O) | 0.16 | 0.16 |
| F7_S(O) | 0.13 | 0.37 |
| F14_VA64(O) | 0.37 | 0.57 |
| Compound 1 dry powder | 0.15 | 0.14 |

All the compositions stored under the accelerated condition (40° C./75% RH) for 2 weeks did not show significantly high content loss of Compound 1 or rapid increase of the impurities compared to the those at t=0. In the case of F7_S(O), the content of Compound 1 was a little bit lower than the drug product acceptance criteria (95.0-105.0% label claim). However, considering that the test was done at the accelerated condition, the composition at 25° C./60% RH, which would be in a semisolid state, would be thought to maintain a higher Compound 1 content.

### Example 5: Pharmacokinetic Analysis Using Animals

Pharmacokinetic (PK) analysis was performed using male Beagle dogs of about 7-8 months old. The dogs were administered a single oral dose of F5_S(O) (containing 3 mg/g Compound 1) or F7_S(O) (containing 2 mg/g Compound 1) as prepared in Example 4-1. Six Beagle dogs (average body weight of about 6.9 kg±0.5) were assigned to each composition group. The compositions were diluted with water so that the dose of Compound 1 was 2 mg/dog. As a control, Mekinist Tablet 2 mg was also administered to six Beagle dogs (average body weight of about 6.8 kg±0.5). The animals in all groups were fasted for at least 16 hours before administration and fed 4 hours after administration.

Before administration (0 h) and 0.5, 1, 1.5, 2, 3, 4, 6, 8, 10, 24, 48, 72, and 120 hours after administration (total 14 time points), about 2 mL of whole blood was drawn from the jugular vein of the Beagle dogs and immediately centrifuged at 3,000 rpm for 5 minutes to separate the plasma. The plasma was analyzed for the concentration of Compound 1 with LC-MS/MS. The results are shown in Table 14.

TABLE 14

| | AUC(last), | | AUC(inf), | | | | | | | | | |
|---|---|---|---|---|---|---|---|---|---|---|---|---|
| | ng * h/ml | | ng * h/ml | | Cmax, ng/ml | | Tmax, hr | | T½, hr | | Ratio to Mekinist | |
| Formulations | Mean | SD | Mean | SD | Mean | SD | Mean | SD | Mean | SD | AUC(inf) | Cmax |
| Mekinist tablet 2 mg | 1783.9 | 280.0 | 1875.1 | 291.9 | 93.1 | 13.8 | 2.6 | 1.3 | 34.1 | 6.9 | — | — |
| F5_S(O) | 1945.7 | 240.0 | 2043.6 | 243.3 | 113.3 | 15.0 | 1.3 | 0.5 | 34.6 | 8.8 | 114.6% | 121.7% |
| F7_S(O) | 1867.2 | 308.4 | 1943.0 | 311.1 | 111.2 | 14.7 | 1.6 | 0.5 | 31.7 | 12.1 | 108.9% | 119.5% |

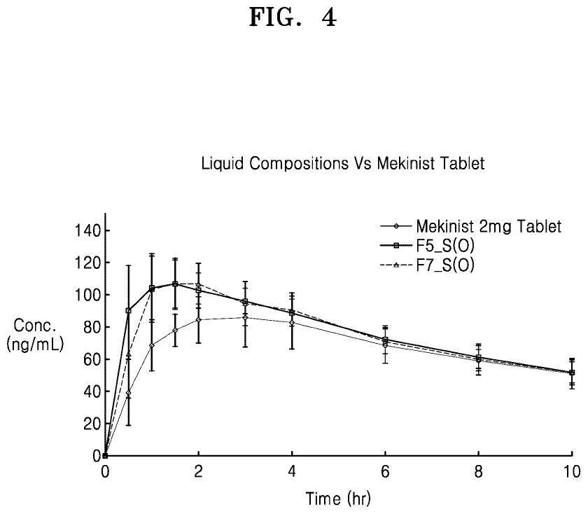

As can be seen from Table 14, both compositions had good pharmacokinetic parameters compared to those of Mekinist. FIG. 4 is a graph showing the PK profile up to 10 hours after administration.

### Example 6: Optimization Study

### Example 6-1. Preparation of Active Compositions

The active compositions as described in Tables 15 and 16 were prepared with consideration of the impact of drug loading and/or the PI agent. The preparation method was the same as described in Example 4-1 except for the content of Compound 1 and the presence of the PI agent.

TABLE 15

| Ingredients | F5(L1) | F5_S(L1) | F5_S(L2) |
|---|---|---|---|
| Polyethylene glycol (PEG400) | 76.596 | 76.135 | 75.944 |
| (weight %) | | | |
| Diethylene glycol monoethyl ether | 20.203 | 20.082 | 20.031 |
| (Transcutol HP) (weight %) | | | |
| Polysorbate (Tween 80-LQ-CQ) | 3.031 | 3.013 | 3.005 |
| (weight %) | | | |
| Polyvinyl caprolactam-polyvinyl | — | 0.600 | 0.800 |
| acetate-polyethylene glycol graft | | | |
| copolymer (Soluplus) (weight %) | | | |
| Butylated hydroxytoluene (BHT) | 0.020 | 0.020 | 0.020 |
| (weight %) | | | |
| Compound 1 (weight %) | 0.150 | 0.150 | 0.200 |
| Compound 1 (mg/g) | 1.500 | 1.500 | 2.000 |

TABLE 16

| Ingredients | F7(L1) | F7_S(L1) | F7_S(L2) |
|---|---|---|---|
| Polyethylene glycol (PEG400) | 34.955 | 34.815 | 34.771 |
| (weight %) | | | |
| Glyceryl monooleate (Peceol) | 4.995 | 4.975 | 4.969 |
| (weight %) | | | |
| Caprylocaproyl polyoxylglyceride | 19.980 | 19.900 | 19.875 |
| (Labrasol ALF) (weight %) | | | |
| Lauroyl polyoxylglyceride (Gelucire | 39.950 | 39.790 | 39.740 |
| 44/14) (weight %) | | | |
| Polyvinyl caprolactam-polyvinyl | — | 0.400 | 0.500 |
| acetate-polyethylene glycol graft | | | |
| copolymer (Soluplus) (weight %) | | | |
| Butylated hydroxytoluene (weight %) | 0.020 | 0.020 | 0.020 |
| Compound 1 (weight %) | 0.100 | 0.100 | 0.125 |
| Compound 1 (mg/g) | 1.000 | 1.000 | 1.250 |

### Example 6-2. In-Vial Stability Test

The six active compositions prepared in Example 6-1 were tested for in-vial stability. The active compositions and control compositions (same as the active composition with the exception of not containing Compound 1) were stored in 1.5 mL glass vials for 24 weeks under the 3 conditions below. The appearance was observed before storage (t=0) and 4 weeks (t=4w), 8 weeks (t=8w), 12 weeks (t=12w), and 24 weeks (t=24w) after storage, and the content of Compound 1 and total impurities were measured with HPLC.

$$25 °C \pm 2 °C and 60 \% RH \pm 5 \% RH$$

$$30 °C \pm 2 °C and 65 \% RH \pm 5 \% RH$$

$$40 °C \pm 2 °C and 75 \% RH \pm 5 \% RH$$

The other conditions and methods were the same as described in Example 4.

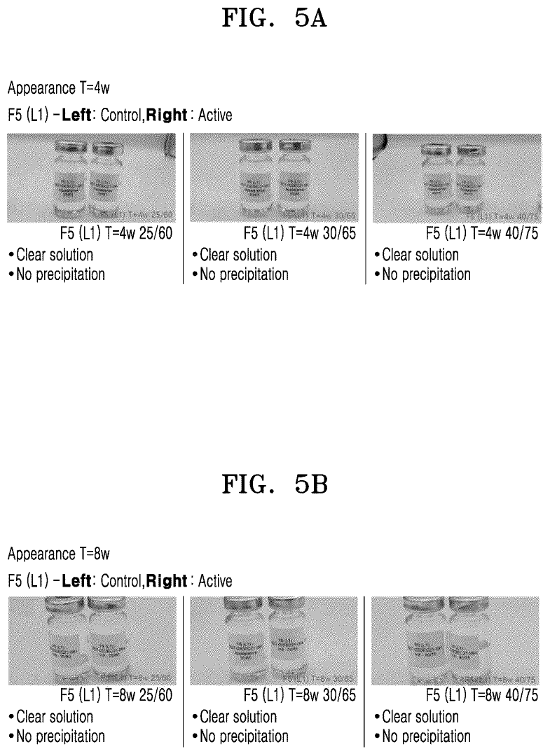

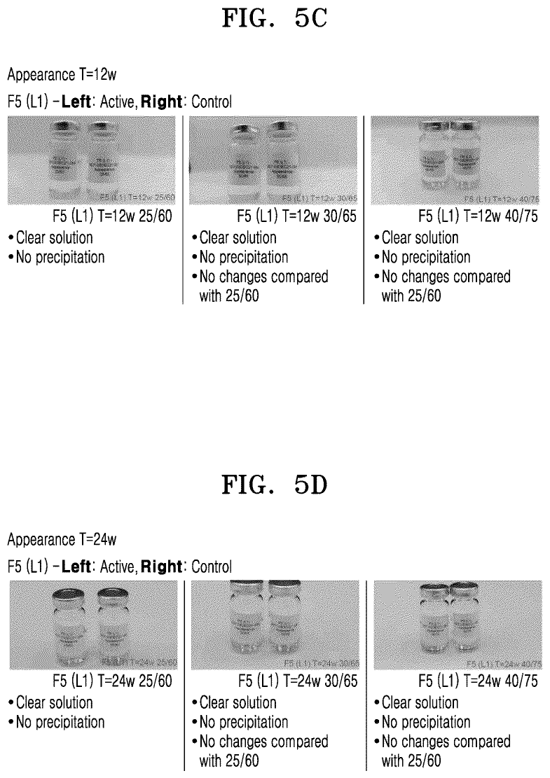

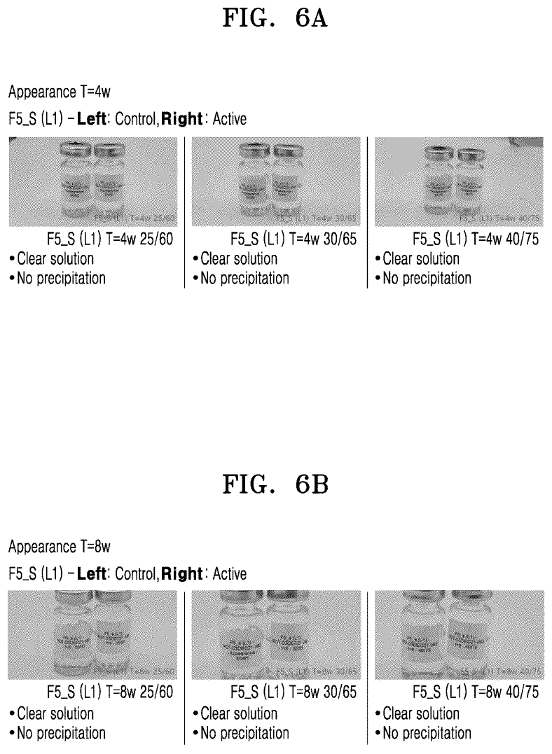

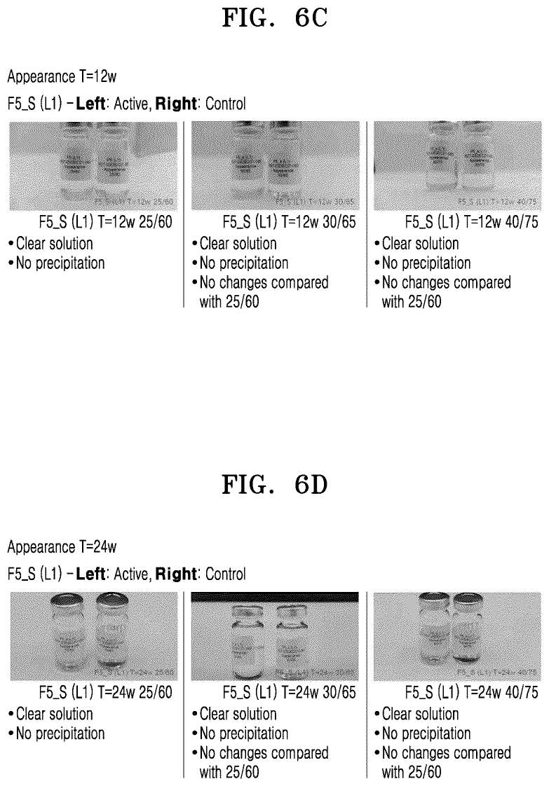

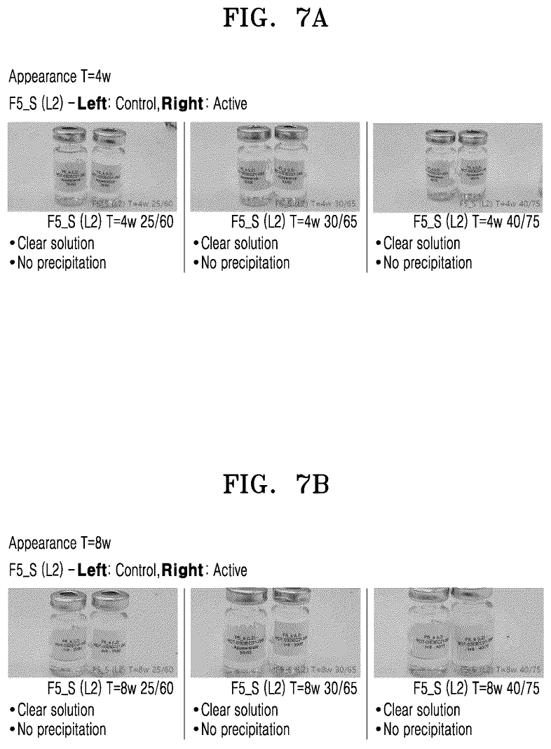

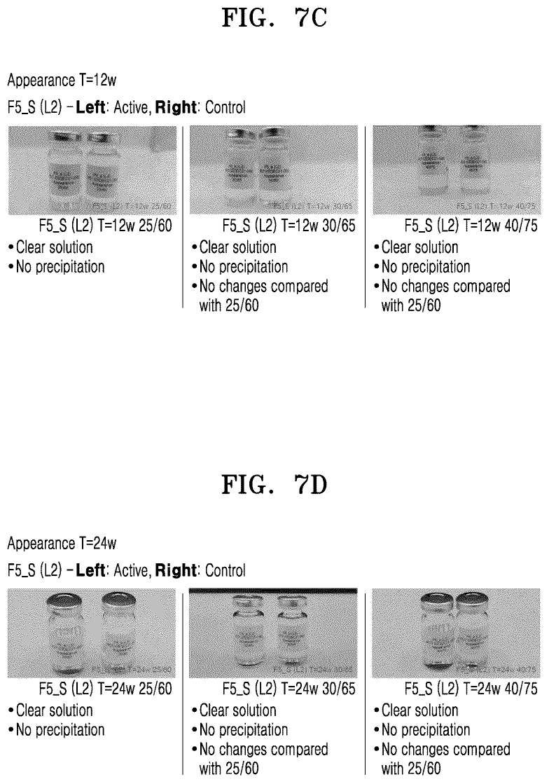

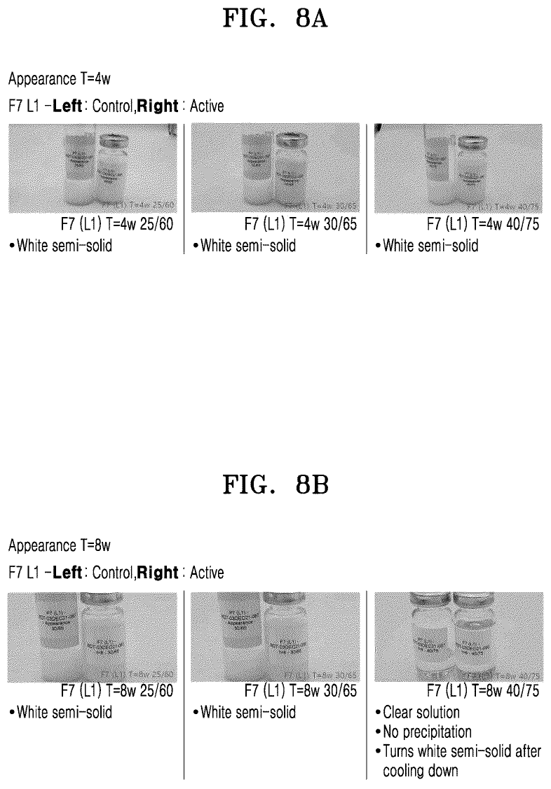

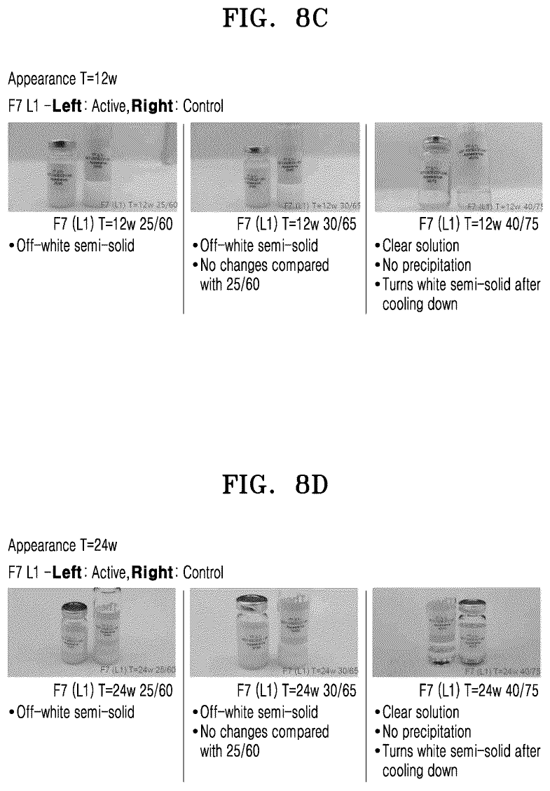

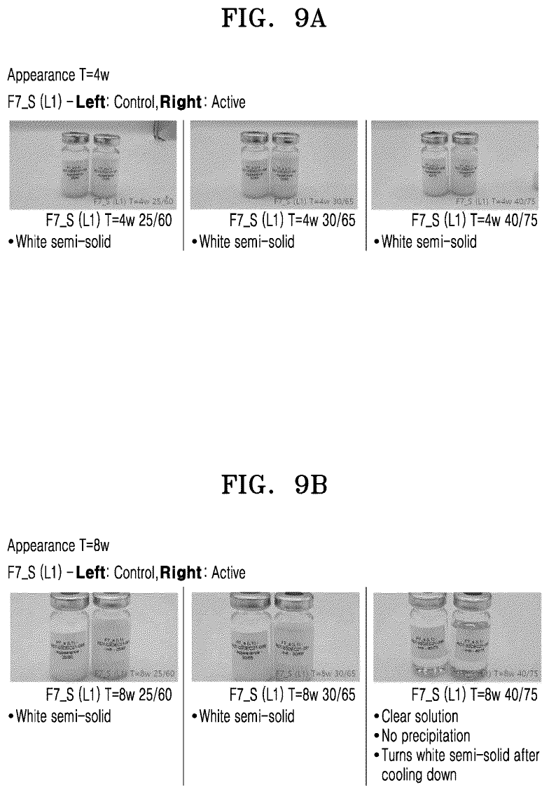

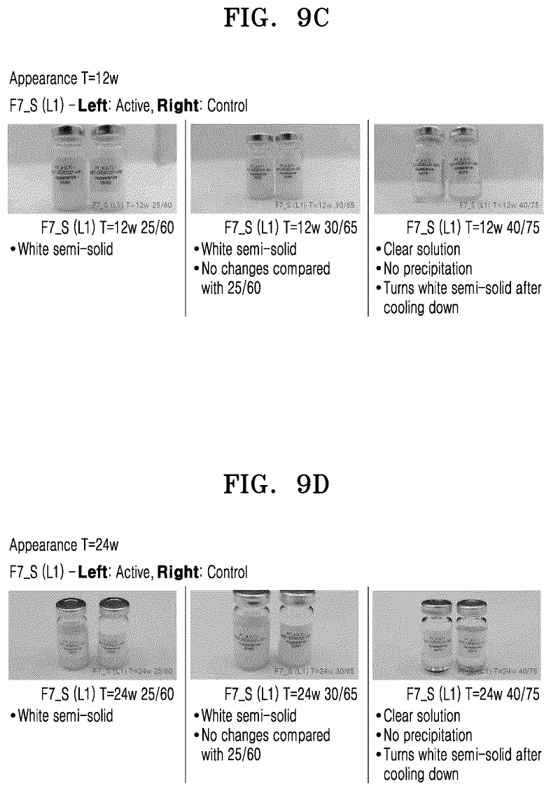

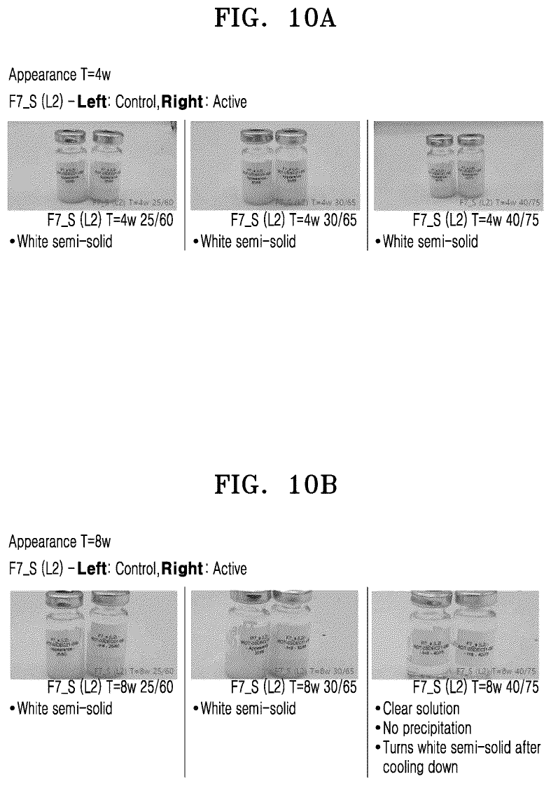

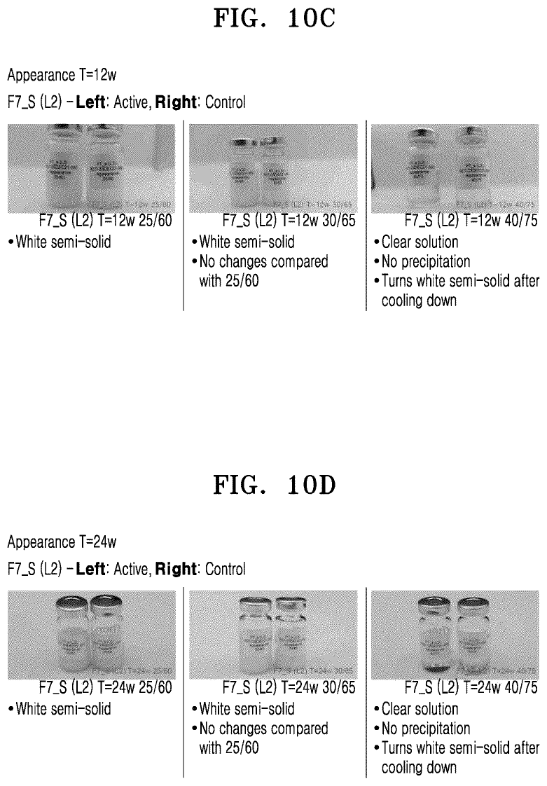

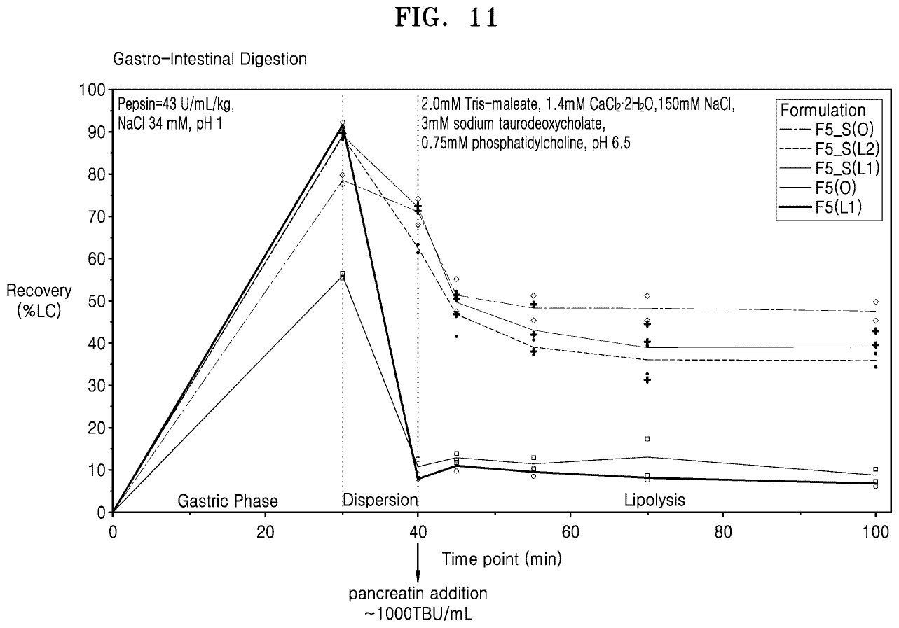

FIGS. 5A to 10D show the results of the observation of appearance. All 6 active compositions were stable for 24 weeks under the 3 conditions above (even at the accelerated condition) without remarkable change of color or appearance of precipitate. The content of Compound 1 and the total impurities measured are described in Tables 17 and 18, respectively.

TABLE 17

| Compound 1 | | | | | | | | | | | | | |
|---|---|---|---|---|---|---|---|---|---|---|---|---|---|
| (% Recovery) | | 25° C./60% RH | | | | 30° C./65% RH | | | | 40° C./75% RH | | | |
| Composition | t = 0 | 4 w | 8 w | 12 w | 24 w | 4 w | 8 w | 12 w | 24 w | 4 w | 8 w | 12 w | 24 w |
| F5(L1) | 99.9 | 96.1 | 100.2 | 101.3 | 101.0 | 96.6 | 100.1 | 100.3 | 100.7 | 95.8 | 99.9 | 99.4 | 99.7 |
| F5_S(L1) | 99.9 | 96.3 | 100.3 | 100.9 | 100.8 | 97.0 | 100.2 | 99.8 | 100.2 | 96.0 | 99.6 | 99.8 | 99.5 |
| F5_S(L2) | 100.2 | 96.5 | 100.6 | 101.1 | 101.2 | 96.4 | 100.5 | 100.4 | 99.2 | 95.7 | 99.7 | 99.4 | 98.9 |
| F7(L1) | 100.0 | 96.8 | 99.0 | 102.8 | 100.2 | 96.0 | 99.8 | 101.3 | 100.4 | 95.7 | 101.4 | 98.7 | 99.5 |
| F7_S(L1) | 100.1 | 97.5 | 98.8 | 99.9 | 99.4 | 96.0 | 98.8 | 99.6 | 98.6 | 95.4 | 97.8 | 99.7 | 99.6 |
| F7_S(L2) | 99.6 | 95.6 | 98.8 | 101.5 | 99.8 | 95.1 | 98.7 | 100.3 | 99.3 | 95.0 | 99.3 | 98.9 | 98.7 |

TABLE 18

| Total impurities | | | | | | | | | | | | | |
|---|---|---|---|---|---|---|---|---|---|---|---|---|---|
| (% label claim) | | 25° C./60% RH | | | | 30° C./65% RH | | | | 40° C./75% RH | | | |
| Composition | t = 0 | 4 w | 8 w | 12 w | 24 w | 4 w | 8 w | 12 w | 24 w | 4 w | 8 w | 12 w | 24 w |
| F5(L1) | 0.19 | 0.19 | 0.19 | 0.17 | 0.19 | 0.19 | 0.19 | 0.16 | 0.26 | 0.19 | 0.23 | 0.25 | 0.31 |
| F5_S(L1) | 0.18 | 0.18 | 0.18 | 0.17 | 0.18 | 0.18 | 0.19 | 0.17 | 0.25 | 0.18 | 0.19 | 0.25 | 0.31 |
| F5_S(L2) | 0.18 | 0.18 | 0.17 | 0.17 | 0.30 | 0.18 | 0.19 | 0.15 | 0.33 | 0.18 | 0.17 | 0.25 | 0.40 |
| F7(L1) | 0.24 | 0.24 | 0.15 | 0.25 | 0.31 | 0.24 | 0.23 | 0.27 | 0.40 | 0.24 | 0.19 | 0.42 | 0.59 |
| F7_S(L1) | 0.25 | 0.25 | 0.19 | 0.18 | 0.30 | 0.25 | 0.19 | 0.24 | 0.34 | 0.25 | 0.22 | 0.33 | 0.56 |
| F7_S(L2) | 0.26 | 0.26 | 0.17 | 0.19 | 0.28 | 0.26 | 0.16 | 0.23 | 0.31 | 0.26 | 0.19 | 0.28 | 0.53 |

All the active compositions showed % recovery of Compound 1 within 95.0-105.0% label claim for 24 weeks under all 3 conditions above (Table 17). Furthermore, there was no rapid increase of impurities for 24 weeks under all 3 conditions above (Table 18), and the individual impurities were maintained below the Identification Threshold (0.5%) of the ICH (International Council for Harmonisation). These results show that the 6 liquid compositions tested have excellent physical and chemical stability.

### Example 7: In Vitro Dispersion Testing in Simulated Gastric Fluid (SGF)

The active compositions were tested for in vitro dispersion performance in simulated gastric fluid (SGF) simulating the condition of gastric fluid to assess their stability (or solubility or suitability) as an oral dosage form. The test was conducted in a USP dissolution apparatus type 2 equipped with a paddle as a stirring element set at 100 rpm filled with SGF (pepsin activity=43 U/mL/Kg, NaCl 34 mM, pH 1). The test was started by the addition of 1 g of the active composition into 300 mL SGF solution at 37.0° C.±0.5° C. After stirring for 30 minutes at 100 rpm, aliquot samples (approximately 2 mL) were taken and centrifuged at 37.0° C.±0.5° C. and 14,000 rpm for 15 minutes. Aliquot samples of supernatant were taken, diluted with a proper diluent, filtered through 0.45 μm PTFE, and assayed for Compound 1 content with HPLC. Test was performed for the samples before storage (t=0) and after storage at 25° C.±60% RH for 4 weeks (t=4w), 8 weeks (t=8w), and 12 weeks (t=12w). Each sample was tested in duplicate. The results are shown in Table 19.

TABLE 19

| Compound 1 (% Recovery) | | | | |
|---|---|---|---|---|
| Active | | 25° C. ± 60% RH | | |
| composition | t = 0 | 4 w | 8 w | 12 w |
| F5(L1) | 91.7% | 88.3% | 94.8% | 91.6% |
| F5_S(L1) | 88.9% | 85.5% | 91.5% | 86.6% |
| F5_S(L2) | 88.5% | 84.8% | 90.0% | 86.8% |
| F7(L1) | 93.0% | 92.5% | 97.2% | 94.3% |
| F7_S(L1) | 92.6% | 92.3% | 96.1% | 94.3% |
| F7_S(L2) | 92.2% | 93.3% | 95.7% | 94.2% |

All the tested active compositions were stable in SGF with no significant change for 12 weeks compared to t=0. In addition, all the active compositions showed the % recovery of Compound 1 to be higher than 85% (in the case of lipid based F7 composition >90%), confirming their good dispersion/emulsion stability in the gastric fluid. This shows that the tested active compositions taken in an oral dosage form would exhibit a good dissolution property even at the low gastric pH condition and in the presence of the digestive fluid. Meanwhile, the presence or absence of a PI agent did not seem to affect the recovery of Compound 1 or the performance of the active compositions in the gastric fluid.

### Example 8: In Vitro Lipolysis Test

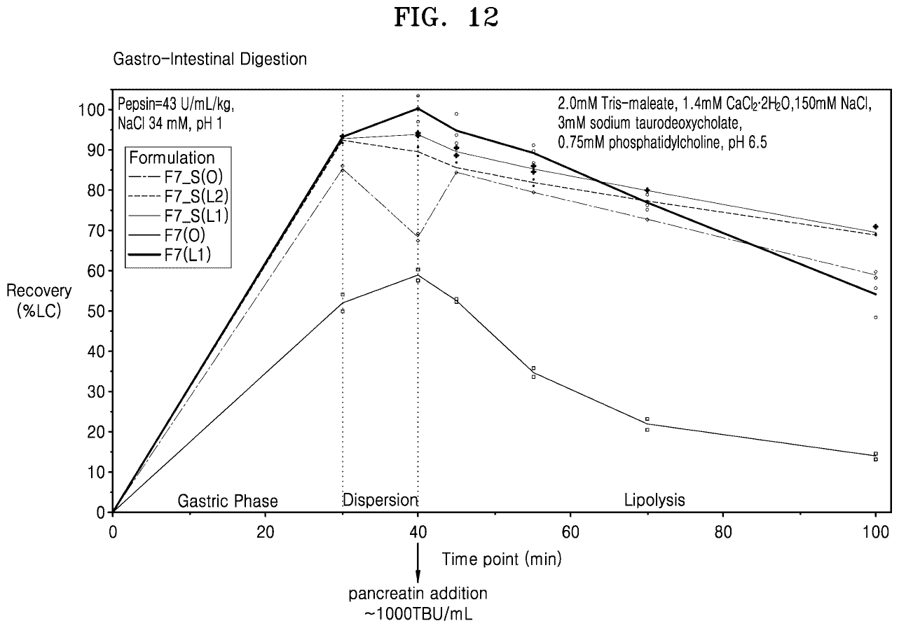

The in vitro lipolysis test was performed with the active compositions prepared in the example above to evaluate the extent of digestion of the compositions and drug distribution between the phases (aqueous, oil, pellet) present in the gastrointestinal tract. The test was conducted using a pH-stat titration apparatus (Metrohm Titrando) connected to a computer and filled with lipolysis buffer at pH 6.5 (2.0 mM Tris-maleate, 1.4 mM CaCl₂·2H₂O, 150 mM NaCl, 3 mM sodium taurodeoxycholate, and 0.75 mM phosphatidylcholine). Approximately 1 g of active composition was dispersed in 36 mL lipolysis buffer at 37.0° C.±0.5° C., then digestion was initiated by addition of 4 mL pancreatin solution (˜1000 TBU/mL). 1 mL aliquot was collected prior to enzyme addition (t=0), then aliquot samples were withdrawn at regular intervals: t=5, 15, 30, and 60 minutes. The lipolysis reaction was quenched immediately after the collection of each sample by addition of 10 μL of 0.5 M 4-bromophenylboronic acid solution (inhibitor). The collected samples were centrifuged for 30 min at 14,000 rpm at 37.0° C., and the aqueous phase was analyzed by HPLC for Compound 1 content. Each composition was tested in duplicate. The results are shown in FIGS. 11 and 12.

FIGS. 11 and 12 are combined graphs of the lipolysis profile and the dispersion profile in SGF measured in Example 7 for the F5 series active compositions and the F7 series lipid-based compositions, respectively. As can be seen from FIG. 11, the F5 series hydrophilic compositions generally had good % recovery in the gastric condition, while the compositions without a PI agent (F5(O) and F5 (L1)) had low % recovery in the intestinal condition. Thus, a PI agent seems necessary for the F5 series hydrophilic compositions to prevent precipitation of Compound 1 and increase the % recovery in the gastrointestinal tract. F5_S(O), F5_S (L1), and F5_S (L2) containing a PI agent showed stable % recovery despite the large pH change from gastric pH 1 to intestinal pH 6.5 and the presence of digestive fluid. Among the F7 series lipid-based compositions, F7(O) that contains 2 mg/g Compound 1 and no PI agent showed the lowest % recovery (FIG. 12). However, addition of a PI agent (F7_S(O)) increased the % recovery by 3˜4 folds. F7(L1), F7_S (L1), and F7_S (L2) containing less than 2 mg/g of Compound 1 showed good recovery regardless of the presence or absence of a PI agent. These results suggest that upon oral administration, the active compositions prepared in the examples can maintain stable and good drug concentration even under the changing conditions throughout the gastrointestinal tract.

### Example 9: Preparation of Capsules

TABLE 20

| Ingredients | CF7(L1) | CF7_S(L1) |
|---|---|---|
| Polyethylene glycol (PEG400) | 174.790 | 174.090 |
| (mg) | | |
| Glyceryl monooleate (Peceol) (mg) | 24.970 | 24.870 |
| Caprylocaproyl polyoxylglyceride | 99.880 | 99.480 |
| (Labrasol ALF) (mg) | | |
| Lauroyl polyoxylglyceride | 199.760 | 198.960 |
| (Gelucire 44/14) (mg) | | |
| Polyvinyl caprolactam-polyvinyl | — | 2.000 |
| acetate-polyethylene glycol graft | | |
| copolymer (Soluplus) (mg) | | |
| Butylated hydroxytoluene (mg) | 0.100 | 0.100 |
| Compound 1 (mg) | 0.500 | 0.500 |
| Total (mg) | 500 | 500 |

The liquid fill compositions described in Table 20 above were prepared in a similar manner as in Example 4-1. A base gel solution was prepared by mixing gelatin, glycerol, and water, and then titanium dioxide and yellow iron oxide each dispersed in glycerol were added to the base gel solution to prepare a film solution. The liquid fill compositions were encapsulated using a rotary die encapsulation machine, and the resulting capsules were dried to obtain soft capsules.

### Example 10: Preparation of Liquid Preparation for Oral Administration

TABLE 21

| Ingredients | LF5_S(L1) | LF7_S(L1) |
|---|---|---|
| Polyethylene glycol (PEG400) | 2537.83 | 1740.75 |
| (mg) | | |
| Diethylene glycol monoethyl ether | 669.40 | — |
| (Transcutol HP) (mg) | | |
| Polysorbate (Tween 80-LQ-CQ) | 100.43 | — |
| (mg) | | |
| Glyceryl monooleate (Peceol) (mg) | — | 248.75 |
| Caprylocaproyl polyoxylglyceride | — | 995.00 |
| (Labrasol ALF) (mg) | | |
| Lauroyl polyoxylglyceride | — | 1989.50 |
| (Gelucire 44/14) (mg) | | |
| Polyvinyl caprolactam-polyvinyl | 20.00 | 20.00 |
| acetate-polyethylene glycol graft | | |
| copolymer (Soluplus) (mg) | | |
| Butylated hydroxytoluene (mg) | 0.67 | 1.00 |
| Compound 1 (mg) | 5.00 | 5.00 |
| Purified water | q.s. | q.s. |
| Total (mL) | 100.00 | 100.00 |

Liquid compositions were prepared with the ingredients in Table 21 above, except for the purified water, in a method similar to that described in Example 4-1. An appropriate amount of purified water was added to the liquid composition to make the final volume of the oral liquid preparation 100 mL. The mixture was stirred until completely dispersed to obtain 100 ml of an oral liquid preparation with a final concentration of Compound 1 of 0.05 mg/mL. The oral liquid preparation can be divided into multiple doses to adjust the daily dosage of Compound 1.

1. A liquid pharmaceutical composition comprising:
(a) a drug, which is N-(3-{3-Cyclopropyl-5-[(2-fluoro-4-iodophenyl)amino]-6,8-dimethyl-2,4,7-trioxo-3,4,6, 7-tetrahydropyrido[4,3-d]pyrimidin-1 (2H)-yl}phenyl) acetamide (“compound”) or a pharmaceutically acceptable salt or solvate thereof, and
(b) pharmaceutically acceptable solubilizers,
wherein the solubilizers consist of, based on the total weight of the solubilizers, 0 to 20 weight % of one or more oils, 0 to 30 weight % of one or more water-insoluble surfactants having an HLB of less than 11, 50 to 70 weight % of one or more water-soluble surfactants having an HLB of 11 or greater, and 10 to 40 weight % of one or more hydrophilic cosolvents, wherein at least one of the oil and the water-insoluble surfactant is included as an essential component;
the drug is included at the compound content of 0.01 weight % to 0.2 weight %, 0.01 weight % to 0.125 weight %, or 0.05 weight % to 0.1 weight %, based on the total weight of the composition;
the oil is vegetable oil, medium chain triglyceride (MCT) oil, or mono-, di- or tri-glyceride of C₁₈-C₂₀ unsaturated fatty acid;
the water-insoluble surfactant is phospholipid, lecithin, phosphatidylcholine, sorbitan fatty acid ester, propylene glycol fatty acid ester, glycerol fatty acid ester, or polyoxylglyceride;
the water-soluble surfactant is polyoxyl castor oil, polyoxyl hydrogenated castor oil, polyoxyl sorbitan fatty acid ester, polyoxyl tocopherol ester derivatives, or polyoxylglyceride; and
the hydrophilic cosolvent is diethylene glycol monoethyl ether, propylene glycol, polyethylene glycol, or alcohol.

2. The pharmaceutical composition according to claim 1, wherein the oil is at least one selected from the group consisting of glyceryl monooleate, medium chain triglyceride oil, soybean oil, and glyceryl monolinoleate.

3. The pharmaceutical composition according to claim 1, wherein the water-insoluble surfactant is at least one selected from the group consisting of sorbitan monooleate, phosphatidylcholine in propylene glycol, phosphatidylcholine in medium chain triglyceride oil, lecithin, glyceryl monocaprylocaprate, propylene glycol monocaprylate, and oleoyl polyoxylglyceride.

4. The pharmaceutical composition according to claim 1, wherein the water-soluble surfactant is at least one selected from the group consisting of polyoxyl 35 castor oil, polyoxyl 40 hydrogenated castor oil, polysorbate, tocopherol polyethylene glycol succinate, lauroyl polyoxylglyceride, and caprylocaproyl polyoxylglyceride.

5. The pharmaceutical composition according to claim 1, further comprises one or more precipitation inhibitors selected from the group consisting of polyvinyl caprolactam-polyvinyl acetate-polyethylene glycol graft copolymer, polyvinylpyrrolidone, copovidone, hydroxypropyl methylcellulose (HPMC), and hydroxypropyl methylcellulose acetate succinate (HPMCAS).

6. The pharmaceutical composition according to claim 5, wherein the precipitation inhibitor is polyvinyl caprolactam-polyvinyl acetate-polyethylene glycol graft copolymer (Soluplus).

7. The pharmaceutical composition according to claim 5, wherein the weight ratio of the compound to the precipitation inhibitor is in the range of 1:1 to 1:10, 1:4 to 1:9, or 1:4.

8. The pharmaceutical composition according to claim 1, wherein the solubilizers consist of, based on the total weight of the solubilizers,
(a) 5 weight % of one or more oils, 60 weight % of one or more water-soluble surfactants, and 35 weight % of one or more hydrophilic cosolvents;
(b) 30 weight % of one or more water-insoluble surfactants, 55 weight % of one or more water-soluble surfactants, and 15 weight % of one or more hydrophilic cosolvents;
(c) 20 weight % of one or more water-insoluble surfactants, 66 weight % of one or more water-soluble surfactants, and 14 weight % of one or more hydrophilic cosolvents;
(d) 25 weight % of one or more water-insoluble surfactants, 60 weight % of one or more water-soluble surfactants, and 15 weight % of one or more hydrophilic cosolvents; or
(e) 5 weight % of one or more oils, 20 weight % of one or more water-insoluble surfactants, 60 weight % of one or more water-soluble surfactants, and 15 weight % of one or more hydrophilic cosolvents.

9. The pharmaceutical composition according to claim 1, wherein the solubilizers consist of, based on the total weight of the solubilizers, 5 weight % of glyceryl monooleate, 35 weight % of polyethylene glycol 400 (PEG400), 20 weight % of caprylocaproyl polyoxylglyceride, and 40 weight % of lauroyl polyoxylglyceride, and the drug is included at the compound content of 0.01 weight % to 0.2 weight %, 0.01 weight % to 0.125 weight %, or 0.05 weight % to 0.1 weight %, based on the total weight of the composition.

10. The pharmaceutical composition according to claim 9, further comprises one or more precipitation inhibitors.

11. The pharmaceutical composition according to claim 1, further comprising one or more antioxidants.

12. The pharmaceutical composition according to claim 1, wherein the composition is for oral administration.

13. A liquid pharmaceutical composition comprising:
(a) a drug, which N-(3-{3-Cyclopropyl-5-[(2-fluoro-4-iodophenyl)amino]-6,8-dimethyl-2,4, 7-trioxo-3,4,6, 7-tetrahydropyrido[4,3-d]pyrimidin-1 (2H)-yl}phenyl) acetamide (“compound”) or a pharmaceutically acceptable salt or solvate thereof,
(b) pharmaceutically acceptable solubilizers, and
(c) one or more precipitation inhibitors,
wherein the solubilizers consist of, based on the total weight of the solubilizers, 3 weight % of one or more water-soluble surfactants having an HLB of 11 or greater, and 97 weight % of one or more hydrophilic cosolvents;
the drug is included at the compound content of 0.01 weight % to 0.3 weight %, 0.01 weight % to 0.2 weight %, or 0.05 weight % to 0.15 weight %, based on the total weight of the composition;
the water-soluble surfactant is polyoxyl castor oil, polyoxyl hydrogenated castor oil, polyoxyl sorbitan fatty acid ester, polyoxyl tocopherol ester derivatives, or polyoxylglyceride;
the hydrophilic cosolvent is diethylene glycol monoethyl ether, propylene glycol, polyethylene glycol, or alcohol; and
the precipitation inhibitor is polyvinyl caprolactam-polyvinyl acetate-polyethylene glycol graft copolymer, polyvinylpyrrolidone, copovidone, hydroxypropyl methylcellulose (HPMC), or hydroxypropyl methylcellulose acetate succinate (HPMCAS).

14. The pharmaceutical composition according to claim 13, wherein the water-soluble surfactant is at least one selected from the group consisting of polyoxyl 35 castor oil, polyoxyl 40 hydrogenated castor oil, polysorbate, tocopherol polyethylene glycol succinate, lauroyl polyoxylglyceride, and caprylocaproyl polyoxylglyceride.

15. The pharmaceutical composition according to claim 13, wherein the weight ratio of the compound to the precipitation inhibitor is in the range of 1:1 to 1:10, 1:4 to 1:9, or 1:4.

16. The pharmaceutical composition according to claim 13, wherein the precipitation inhibitor is polyvinyl caprolactam-polyvinyl acetate-polyethylene glycol graft copolymer (Soluplus).

17. The pharmaceutical composition according to claim 13:
wherein the solubilizers consist of, based on the total weight of the solubilizers, 77 weight % of PEG400, 20 weight % of diethylene glycol monoethyl ether, and 3 weight % of Polysorbate 80,
and the drug is included at the compound content of 0.01 weight % to 0.3 weight %, 0.01 weight % to 0.2 weight %, or 0.05 weight % to 0.15 weight %, based on the total weight of the composition.

18. The pharmaceutical composition according to claim 13, further comprising one or more antioxidants.

19. The pharmaceutical composition according to claim 13, wherein the composition is for oral administration.
